(19) United States
(12) Patent Application Publication
(10) Pub. No.: US 2008/0027330 A1
(43) Pub. Date: Jan. 31, 2008

(54) RISK ASSESSMENT METHOD FOR ACUTE CARDIOVASCULAR EVENTS

(75) Inventors: Morteza Naghavi, Houston, TX (US); Timothy J. O'Brien, Anoka, MN (US); Craig Jamieson, Houston, TX (US); Mark C. Johnson, Houston, TX (US); Naser Ahmadi, Torrance, CA (US); Haider A. Hassan, Houston, TX (US)

Correspondence Address: WONG, CABELLO, LUTSCH, RUTHERFORD & BRUCCULERI,;L.L.P., 20333 SH 249, SUITE 600, HOUSTON, TX, 77070, US

(73) Assignee: ENDOTHELIX, INC., Houston, TX (US)

(21) Appl. No.: 11/749,105

(22) Filed: May 15, 2007

Related U.S. Application Data
(63) Continuation-in-part of application No. 11/690,122, filed on Mar. 22, 2007.
(60) Provisional application No. 60/747,276, filed on May 15, 2006.

(51) Int. Cl. A61B 5/02

(52) U.S. Cl. 600/481

(57) ABSTRACT

Methods and apparatus are provided for assessing the risk of an acute cardiovascular event that includes providing an endothelial or vascular function test to identify higher risk from lower risk individuals in a population of symptomatic individuals presenting with chest pain that have inconclusive results in ECG and cardiovascular marker tests, such as a tropinin test, and are administered for triage in hospital and additional tests such as ECG exercise and nuclear stress tests. The invention further provides methods and apparatus for assessing the vascular status and response of patients in clinical trials for cardiovascular therapies.

### CROSS REFERENCE TO RELATED APPLICATIONS

This application claims priority under 35 USC §119 to U.S. Provisional Application No. 60/747,276, filed May 15, 2006, the disclosure of which is incorporated herein by reference in its entirety. This application also claims priority as a continuation-in-part application to U.S. patent application Ser. No. 11/690,122, filed Mar. 22, 2007, the disclosure of which is incorporated by reference in its entirety.

### FIELD OF THE INVENTION

This invention relates to methods for assessing the risk of an imminent heart attack and to characterization of cardiovascular status for inclusion in clinical trials and in selection of therapy.

### BACKGROUND OF THE INVENTION

Without limiting the scope of the invention, its background is described in connection with novel methods for assessing the risk that an individual is likely to have an acute cardiovascular event such as a heart attack or stroke. Heart attacks and strokes are an ultimate and acute manifestation of underlying cardiovascular disease (CVD) which is largely due to atherosclerotic processes. CVD is the leading cause of death in the United States and most developed countries and is a fast growing problem in the developing countries as well. Often sudden coronary death is the first sign of CVD. Following an acute coronary event, individuals are at high risk for a subsequent event. The unpredictable nature of heart attack and the need for cost-effective screening in large groups of asymptomatic at-risk populations are unsolved problems in cardiovascular healthcare.

In the past 50 years, although numerous risk factors for atherosclerosis have been reported, the ability to predict a cardiovascular event, particularly in the near term, remains elusive. Numerous population studies have shown that over 90% of CVD patients have one or more risk factors (high cholesterol, blood pressure, smoking, diabetes etc.). However, 70-80% of the non-CVD population also have one or more risk factors. Indeed over 200 risk factors have been reported, including a number of emerging serologic markers. Presently, lipid profiling (Total LDL, HDL, homocysteine, and, to a lesser degree, C-Reactive Protein (CRP)), have been adapted for coronary risk assessment. A recent guideline has brought to light the need for direct and individualized assessment of vascular health beyond risk factors. (Naghavi et al. From Vulnerable Plaque to Vulnerable Patient. Executive Summary of the Screening for Heart Attack Prevention and Education (SHAPE) Task Force Report. The American J. of Cardiology. Supplement to vol 98, no. 2. Jul. 17, 2006). In short, the predictive accuracy of risk factor analysis in a given individual is poor. The SHAPE Guideline highlights the need for structural and functional assessment of arterial system in addition to risk factor analysis but recognizes insufficiencies in available tools for functional assessment of atherosclerosis.

One focus of functional cardiovascular system assessment has been the endothelial system. Endothelial function (EF) is accepted as the most sensitive indicator of vascular function. EF has been labeled a “barometer of cardiovascular risk” and is well-recognized as the target organ of cardiovascular disease. Endothelial cells form the lining of the vasculature. In addition to this barrier function, endothelial cells play a central role in multiple regulatory systems including vasomotion, inflammation, thrombosis, tissue growth and angiogenesis. When there is increased demand for blood by certain organs of the body, endothelial cells release nitric oxide (NO), which increases the diameter of arteries and thereby increases blood flow. NO release is important not only for the regulation of vascular tone but also for the modulation of cardiac contractility, vessel injury and the development of atherosclerosis. Presence of atherosclerosis hampers the normal functioning of these cells, blocking NO-mediated vasodilation and making the arteries stiffer and less able to expand and contract. The loss of ability of an artery to respond to increased and sudden demand is called endothelial dysfunction (EDF).

Endothelial dysfunction is the target organ damaged in association with essentially all of the cardiovascular risk factors and endothelial failure is the end stage that leads to clinical events in cardiovascular disease. Numerous experimental, clinical, and epidemiologic studies have shown that endothelial function is altered in presence of established risk factors such as hypertension, hypercholesterolemia, diabetes mellitus and emerging risk factors such as hyperhomocysteinemia, CRP, and fibrinogen. Evidence showing strong correlations between endothelial dysfunction and other sub-clinical markers of atherosclerosis such as carotid intima media thickness, coronary calcium score and ankle brachial index has also emerged. More importantly endothelial dysfunction has been reported to be predictive of coronary, cerebro-vascular and peripheral arterial disease and can be detected before the development of angiographically significant plaque formation in the coronary and peripheral vasculature by measuring the response to pharmacological and physiological stressors. Endothelial function not only predicts risk it also tracks changes in response to therapy (pharmacologic and non-pharmacologic) and alterations in risk factors.

Traditional invasive techniques for assessment of endothelial function include forearm plethysmography with intra-arterial acetylcholine challenge testing, cold pressor tests by invasive quantitative coronary angiography, and injection of radioactive materials and mapping blood flow by tracing movement of radiation. The invasive nature of these tests limits widespread use, particularly in the asymptomatic population. For purposes of assessing the likelihood that an individual is actually undergoing an acute cardiovascular event such as a heart attack, the traditional invasive techniques for assessment of endothelial function would not provide an increment of cost savings over existing practice.

Non-invasive methods include: measurement of the percent change in diameter of the left main trunk induced by cold pressor test with two-dimensional (2-D) echocardiography; the Dundee step test measuring the blood pressure response of a person to exercise (N Tzemos, et al. Q J Med 95 (2002) 423-429); laser Doppler perfusion imaging and iontophoresis; high resolution B-mode ultrasound to study vascular dimensions (T J Anderson, et al. J. Am. Col. Cardiol. 26(5) (1995) 1235-41); occlusive arm cuff plethysmography (S Bystrom, et al. Scand J Clin Lab Invest 58(7) (1998) 569-76); and digital plethysmography or peripheral arterial tonometry (PAT)(A Chenzbraun et al. Cardiology 95(3) (2001) 126-30). Of these, brachial artery imaging with high-resolution ultrasound (BAUS) during reactive hyperemia is considered the gold standard method of determining peripheral vascular function. Arm cuff inflation provides a suprasystolic pressure stimulus. Ischemia reduces distal resistance and opening the cuff induces stretch in the artery. Imaging of the diameter of the artery along with measuring the peak flow defines endothelial function. However, this method requires very sophisticated equipment and operators that are only available in a few specialized laboratories worldwide. Thus, despite widespread use of BAUS in clinical research, technical challenges, poor reproducibility, and considerable operator dependency have limited the use of this technique to vascular research laboratories and do not commend this test for an assessment of the probability that an individual in undergoing an acute cardiovascular event.

Every year over 6 million people visit emergency rooms in the United States because of chest pain or discomfort but a heart attack (Acute Myocardial Infarction, “AMI”, or Acute Coronary Syndrome, “ACS”) or serious cardiovascular related issue is present in less than 1.5 million of such patients. Due to the fatal nature of a heart attack, a prompt and detailed work-up with its attendant cost is conducted to assure all potential cases of heart attack receive the needed emergency care. Over time, a battery of techniques and methods including bedside and laboratory tests have been used to evaluate chest pain and stratify patients into high, intermediate or low risk of present or impending AMI.

Currently, the stepwise algorithm for screening individuals with chest pain includes a detailed history of characteristics of the chest pain followed by an electrocardiogram and a blood test. If the ECG is positive for ST elevation (ST-Segment Elevation MI or “STEMI” patient), the patient is sent immediately to the cardiac catherization (“cath”) lab and/or for thrombolytic therapy.

However, if ECG findings are negative (no ST elevation), a series of tests are conducted to differentiate high risk Non-ST-Segment Elevation Myocardial Infarction (“NSTEMI”) patients from low risk patients. This stratification begins with blood tests (biochemical markers) of myocardial injury. Several cardiac biochemical markers are available including Creatine Kinase-MB (CK-MB) and CK-MB isoforms, myoglobin and cardiac tropinins (including cTnI and cTnT). The cardiac specific troponin test is regarded as the primary cardiac marker for detection of acute coronary syndromes. A cut off value, which varies in different hospitals, is used by emergency rooms to define troponin positive and tropinin negative. A large group of individuals with chest pain are troponin negative at arrival to the emergency room and they are routed to chest pain centers (triage or observation centers in each emergency department) for work-up and subsequent measurement of troponin (every 6 h for 24 hr). If any of troponin tests turns positive the patient is sent to coronary angiography.

Of particular importance in the diagnostic value of cardiac biochemical markers is the fact that all of these markers become elevated only as a consequence of the death of muscle cells. Myoglobin, although suffering from a lack of specificity as to cardiac muscle damage, is one of the earliest markers to be elevated and increases from 2-4 hours after an acute myocardial infarction (AMI) with a peak at 5-9 hours. By contrast, CKMB and the cardiac tropinins increase 4-6 hours post AMI and peak at 10-24 hours.

If the troponin remains negative after 24 hrs, the individual is sent for a treadmill ECG stress test. If the test is negative the patient is discharged. However, a significant number of troponin negative patients are determined to be high risk on the basis of a positive stress test and the patient is sent for additional testing including nuclear imaging stress test and coronary angiography in cath lab. A detailed description of the national guideline for management of chest pain is available on the website of the American College of Cardiology.

As mentioned, more than 70% of the chest pain population is sent home after numerous tests with the diagnosis of no heart attack. This process is a very expensive part of the cardiovascular healthcare system. An average of $5,000 per person can result in over $30 billion in annual costs. Any cost-effective effort for improving the risk assessment is most wanted. A similar observation procedure is made for individuals with symptoms of stroke although with completely different tests and stepwise algorithms. Nonetheless, there remains a need for improvement in risk stratification such that extensive work-ups are properly directed at high risk individuals.

### BRIEF SUMMARY OF THE INVENTION

This disclosure teaches a method for assessing the risk of an acute cardiovascular event that includes providing an endothelial or vascular function test to identify higher risk from lower risk individuals in a population of individuals presenting with chest pain that have inconclusive results in ECG and cardiovascular marker tests, such as a tropinin test, and are administered for triage in hospital and additional tests such as ECG exercise and nuclear stress tests.

In one embodiment of the invention, methods are provided to improve risk assessment for an acute cardiovascular event in a patient presenting with chest pain comprising performing a structural or functional atherosclerosis test on the patient. The functional atherosclerosis test is preferably a non-invasive non-imaging vascular function test that measures vascular response to reactive hyperemia induced by vascular occlusive challenge. In one embodiment, the vascular response to reactive hyperemia is determined on an extremity that is distal to a vascular occlusive challenge by vascular function measurements, at least before and after the vascular occlusive challenge. In one embodiment, the vascular function measurements are serial temperature measurements. The vascular occlusive challenge can be implemented by inflation of a blood pressure cuff on an arm and the serial temperature measurements are made on a finger. The endothelial or vascular function test can be a non-invasive non-imaging vascular function test such as Endothelix's VENDYS DTM test or Doppler test (WO04/17905 and WO05/118516, previously invented by the inventors of this invention and incorporated herein by reference).

In other embodiments, the vascular response to reactive hyperemia is determined on an extremity that is distal to a vascular occlusive challenge by serial Doppler ultrasound measurements, pulse wave velocity measurements, and/or plethysmographic measurements of pulse wave amplitude (such as by Peripheral Arterial Tonometry or PAT), at least before and after the vascular occlusive challenge.

In one embodiment, the functional atherosclerosis test is a non-invasive coronary vasoreactivity imaging test that measures change in coronary flow and/or diameter with provocation. The provocation can be, for example, a cold-pressor test. In one embodiment, the coronary vasoreactivity imaging test is performed by noninvasive echocardiography.

In accordance with the invention, individuals with high likelihood of coronary heart disease but with negative ECG and cardiovascular marker (e.g. troponin) tests can be saved from the additional cost of these tests and their associated costs. In a further embodiment, the cardiovascular risk assessment involves a combination of an endothelial/vascular function test with Framingham Risk Scoring or other risk factor based cardiovascular risk scoring systems.

The method may further include determining a level of a marker of cardiovascular injury and/or a marker of cardiovascular risk. Currently available markers of cardiovascular injury include the cardiac tropinins, CK-MB and CK-MB isoforms, and/or myoglobin. Emerging markers of cardiovascular risk include CRP (C reactive protein), ICAM (inter-cellular adhesion molecule), SAP (serum amyloid P), MPO (myeloperoxidase), ADMA (asymmetric dimethylarginine), NO (nitric oxide), NO compounds/metabolites, and skin sterol (also called skin cholesterol).

In one embodiment, a combination is provided of an endothelial/vascular function test with other tests for atherosclerotic status such coronary calcium scoring or carotid IMT test (measurement of carotid artery intima-media thickness or plaque using ultrasound or MRI). In certain cases the coronary calcium can be seen in a simple chest x-ray.

In other embodiments of the invention a method is provided for identifying a high risk of an acute cardiovascular event in a patient presenting with chest pain including performing an EKG on the patient to determine an ST elevation. If the ST is not elevated, structural and/or functional atherosclerosis test is performed on the patient to determine if further evaluation is required. In one embodiment a cardiovascular risk factor score, such as by Framington Risk Scoring, is additional determined for the patient.

In another embodiment of the invention, a method is provided for characterizing response to therapy in a clinical trial of a medication, device and/or drug that may affect a cardiovascular system function. In one embodiment, the effects of the medication, device and/or drug are determined by characterization of the micro, macrovascular, and/or neurovascular status of the patient at least before and after treatment. In one embodiment, the method includes performing a structural and/or functional atherosclerosis test on the patient, and segregating the patient to a treatment group characterized by patients having similar structural and/or functional atherosclerosis test results. In one embodiment, the functional atherosclerosis (also termed vascular functional) test is a non-invasive non-imaging vascular function test that measures vascular response to reactive hyperemia induced by vascular occlusive challenge, preferably determined on an extremity that is distal to a vascular occlusive challenge by one or more of the following tests performed at least before and after the vascular occlusive challenge: serial temperature measurements, Doppler ultrasound measurements, pulse wave velocity measurements, and plethysmography. The structural arthrosclerosis (also termed vascular structural) test may be a coronary vasoreactivity imaging test performed by noninvasive echocardiography. In one embodiment, a risk factor score for a future acute cardiovascular event is determined and is combined with the structural and/or functional atherosclerosis test results to establish a combined risk score for a future acute cardiovascular event and patients are segregated into treatment groups based on these risk scores.

In another embodiment a method for assessing suitability of a medication, device and/or drug for treatment of a condition affecting a cardiovascular or autonomic nervous system function in an individual patient is provided including the steps of characterizing a structural and/or functional atherosclerotic status of the patient; comparing the characterized atherosclerotic status with clinical trial results of the medication, device and/or therapy in patients having a similar status; and determining whether or not the medication, device and/or therapy is suitable in patients having the characterized atherosclerotic status based on the clinical trial results.

In a further embodiments, a method of risk assessment in a patient presenting with a possible acute cardiovascular symptom is provided including first determining a cardiac specific injury marker level in the patient. If the patient is negative or equivocal for cardiac specific injury markers, one or more tests for atherosclerosis are performed on the patient; and if one or more of the tests for atherosclerosis are positive, the patient is sent to the cath lab for coronary angiography. In one preferred embodiment, a further step of risk factor scoring, such as for example by Framington Risk Factors, is conducted and the risk factor score obtained is combined with the atherosclerosis test result to generate a combined risk score. In one embodiment, if the patient is negative or equivocal for a cardiac specific injury marker, an endothelial function test, structural atherosclerosis test, and a coronary heart disease (CHD) risk assessment are performed on the patient; and if the patient is determined to have two or more of abnormal endothelial function, structural evidence of atherosclerosis or an at-risk score by CHD risk assessment, coronary angiography is performed.

In accordance with an embodiment of the invention, an individual's baseline and reactive functional status are both determined. Baseline functional status is determined in part by measuring blood pressure, which is influenced by the microvasculature, the macrovasculature and the neurovasculature. Baseline status of the macrovasculature is provided by either or both of Pulse Wave Form (PWF) and Pulse Wave Velocity (PWV). In addition, Digital Thermal Monitoring (DTM) has been determined by the present inventors to provide a powerful measure of neuroreactivity. It has been surprisingly found that when a vascular challenge is applied to a target body such as an arm, the corresponding contralateral remote body reacts as instructed by the neurovasculature. Thus, if blood is occluded from a right arm (target body), a normal neurovasulature senses the need for greater perfusion and directs increased blood flow in the contralateral left arm (remote body). If the individual has a healthy microvasculature, the neurovascular instruction to increase blood flow is effective to induce vasodilation in the contralateral microvasculature and an increase blood flow.

In one embodiment, functional assessment of reactive capacity for the individual is determined using Pulse Wave Velocity (PWV) and/or Pulse Wave Flow (PWF) analysis for the macrovasculature after challenge, such as with a chemical or physical vasostimulant. In one embodiment, functional capacity of the microvasculature is determined using Doppler Flow Velocity (DFV) and/or Digital Thermal Monitoring (DTM) subsequent to vascular challenge.

In one embodiment of the invention a modular functional vascular status assessment apparatus is provided including a CPU in electrical communication with and controlling a plurality of vascular function testing modules including a digital thermal monitoring (DTM) module, a cuff management module, a display or recorder; and a Doppler module comprising at lease one Doppler sensor. In further embodiments, wherein the DTM module comprises a plurality of temperature sensors; the cuff management module comprises a plurality of blood pressure cuffs and blood pressure detectors; and/or the Doppler module controls a plurality of Doppler sensors. In one embodiment, at least one Doppler sensor is adapted for measurement of Doppler flow velocity. In other embodiments, at least one Doppler sensor is adapted for pulse wave form (PWF) analysis. In other embodiments, at least two of the plurality of Doppler sensors are adapted to be disposed over a single arterial flow path and at a spaced apart distance sufficient for pulse wave velocity (PWV) measurement and wherein the CPU is programmed to perform PWV analysis. The placement of the sensors may be assisted by the provision of a template or guide for placement of the sensors, on which the sensors may optionally be slidably mounted.

In certain embodiments of the invention, a functional vascular status assessment apparatus is provided that includes a blood pressure cuff in operable association with at least one Doppler sensor array comprising a plurality of Doppler sensors together with a smart Doppler sensor selector that is adapted to monitor signals from each sensor of the array and select the strongest signal providing sensor for signal collection and reporting. The apparatus may further include a computer programmed to perform PWF analysis based on the signal provided by the smart Doppler sensor selector. By computer it is meant a programmable machine.

In one embodiment of the invention a computer implemented method is provided for assessing cardiovascular risk. The method includes receiving results from one or more vascular functional assessments on an individual; placing the results of the functional assessments into a computational dataset corresponding to the individual; receiving a status for each of a plurality of epidemiologic risk factors; placing the status of each epidemiologic risk factor into the computational dataset corresponding to the individual; and computing a combined functional and epidemiologic relative risk for the individual from the dataset corresponding to the individual. In one embodiment the vascular function assessments include one or more of: DTM, BP, PWV, PWF, DFV, CLVR, and ABI. The risk factors include one or more of traditional and emerging risk factors.

In further embodiments, the computer implemented method is optionally further adapted for receiving results from one or more structural assessments on the individual; placing the results of the one or more structural assessments into the computational dataset corresponding to the individual; and computing a combined functional, epidemiologic, and structural relative risk for the individual from the dataset corresponding to the individual. The structural assessments include determination of pathologic changes including one or more of: increased intima medial thickness, atherosclerotic plaque formation and calcium deposits in at least one vascular bed.

In one embodiment the computer implemented method further includes receiving results from one or more serologic assays of a status of circulatory progenitor cells on the individual; placing the results of the one or more serologic assays into the computational dataset corresponding to the individual; and computing a combined functional, epidemiologic, and serologic relative risk for the individual from the dataset corresponding to the individual.

In one embodiment of the invention, a method of determining a neurovascular status for an individual is provided including locating a blood flow sensor on a test site on the individual and establishing a stable baseline blood flow reading at the site; providing a local vascular or neurovascular vasostimulant to a body part of the individual that is contralateral to the test site; determining a temperature response to the vasostimulant; and establishing a neurovascular reactivity assessment for the individual based on a blood flow response at the test site. In further embodiments, an additional blood flow sensor is located on the contralateral site corresponding to the test site, the additional blood flow sensor located on a vascular tree directly affected by the local vasostimulant. Blood flow at the site distal from the local vasostimulant is detected by a technique selected from the group consisting of: DTM, skin color, nail capilloroscopy, fingertip plethysmography, forearm plethysmography, oxygen saturation change, laser Doppler flow, ultrasound Doppler flow measurement, near-infrared spectroscopy measurement, wash-out of induced skin temperature, and peripheral arterial tonometry.

In one embodiment of the invention a functional vascular status assessment apparatus is provides that includes a blood pressure cuff in operable association with at least one Doppler sensor array comprising a plurality of Doppler sensors; and a smart Doppler sensor selector, wherein the selector monitors signals from each sensor of the array and selects a strongest signal providing sensor for signal collection and reporting. The apparatus may further include a computer programmed to perform PWF analysis based on the signal provided by the smart Doppler sensor selector. In one embodiment the Doppler sensor assay is affixed to an inside surface of the cuff such that the sensors are in contact with the skin. By in contact with the skin, it is meant to potentially include an intervening layer of conducting material or gel. In one embodiment, the Doppler sensor array is disposed essentially circumferentially around in the inside surface of the cuff. Alternatively the Doppler sensors are disposed in a local array. In other embodiments, the Doppler sensors are disposed in a longitudinal array.

In one embodiment for measurement on the arm, a plurality of arrays may be employed including one over the brachial artery and another over the radial artery. Likewise, a plurality of arrays can be utilized on a leg. Alternatively, for ABI purposes, an array can be located over the brachial artery and another array located at an ankle for determining the relative blood pressure.

In one embodiment of the invention, a smart Doppler sensor array apparatus adapted for determining maximum Doppler signal from a target cardiovascular system is provided including at least one Doppler sensor array comprising a plurality of Doppler sensors and a smart Doppler sensor selector, wherein the selector monitors signals from each sensor of the array and selects a sensor providing a desired signal intensity and frequency for signal analysis. The array may include sensors resonating at different frequencies providing information at different depths through a tissue. The array may further include sensors positioned at different angles for locating a maximum Doppler blood flow velocity. In one embodiment the target cardiovascular system is selected from the group consisting of: carotid, brachial, femoral, aortic and coronary. FIG. 27 depicts placement of smart Doppler arrays for detection of Doppler flow velocity at these various locations.

In one embodiment, a computer implemented method of risk assessment in a patient presenting with a possible acute cardiovascular symptom is provided including determining results from one or more vascular functional tests on the patient and placing the determined results of the one or more tests into a computational dataset corresponding to the patient; receiving a status for each of a plurality of epidemiologic risk factors and inputting the received status of the epidemiologic risk factors into the computational dataset corresponding to the patient; and computing a combined functional and epidemiologic relative risk for the individual from the dataset corresponding to the patient. The one or more vascular function tests include one or more of: DTM, BP, PWV, PWF, DFV, CLVR, and ABI. Optionally, the computer implemented method may further include receiving results from one or more structural assessments on the patient and placing the results of the one or more structural assessments into the computational dataset corresponding to the patient; and computing a combined functional, epidemiologic, and structural relative risk for the individual from the dataset corresponding to the individual. The structural assessments may include determination of pathologic changes including one or more of: increased intima medial thickness, atherosclerotic plaque formation and calcium deposits in at least one vascular bed.

In one embodiment, methods and apparatus of comprehensive risk assessment are provided that includes at least three components: functional status of the individual, risk factor assessment based on epidemiologic studies, and structural studies of the individual. Functional assessment in accordance with an embodiment of the invention includes information on the status of three compartments: the microvasculature, the macrovasculature and the neurovasculature. The macrovasculature is composed of large and relatively large conduit vessels, such as for example in the arms, the brachial and radial arteries. The microvasculature is made up of resistance vessels, the arterioles and capillaries. The microvasulature is strongly influenced by the neurovascular system.

The present invention contributes new non-invasive methods and apparatus for functional assessment as well as important combinations of the functional assessment with risk factor and structural analysis in the context of an evaluation for determining a probability that a given individual is experiencing an acute cardiovascular event by providing an estimate of the individual's vascular heath status.

It is emphasized that this summary is not to be interpreted as limiting the scope of these inventions which are limited only by the claims herein.

### BRIEF DESCRIPTION OF THE DRAWINGS

FIG. 1 depicts existing risk stratification for patients presenting with chest pain or, more typically in the case of women, chest discomfort.

FIG. 2 depicts the method of risk assessment according to the invention.

FIG. 3 depicts available tests for atherosclerosis.

FIG. 4 depicts the components of a comprehensive assessment of vascular health.

FIG. 5 depicts functional assessment modules provided in one embodiment of the invention.

FIGS. 6A and B depict contributory factors in a DTM response.

FIGS. 7A and 7B depict the measured components of a DTM response.

FIG. 8 provides a block diagram depicting one embodiment of an entire system level design.

FIG. 9 provides a block diagram depicting one embodiment of DTM Module controller.

FIG. 10 provides a block diagram depicting one embodiment of a Cuff Management Module controller.

FIG. 11 depicts a resident GUI application for operating with the system.

FIG. 12 depicts one embodiment of a DTM Module.

FIG. 13 depicts one embodiment of a DTM sensor.

FIG. 14 depicts one embodiment of a Doppler flow velocity sensor.

FIG. 15 depicts results of measuring the response to reactive hyperemia using a Doppler flow velocity sensor.

FIGS. 16A-C depict Doppler arrays for smart Pulse Wave Form (PWF) analysis.

FIG. 17 graphically depicts the generation of a pulse pressure wave in an artery.

FIG. 18A graphically depicts the oscillatory waveform produced by the pressure wave of arterial flow and reflectance. FIG. 18B graphically depicts the oscillatory waveform produced by the pressure wave of arterial flow and reflectance in a healthy artery. FIG. 18C graphically depicts the oscillatory waveform produced by the pressure wave of arterial flow and reflectance in a stiff artery.

FIG. 19 depicts an embodiment for measuring pulse wave velocity with signals received from each of locations A and B.

FIG. 20 depicts Doppler signals from brachial (A) and radial (B) arteries of FIG. 19 overlaid.

FIG. 21A depicts the results of a baseline PWV analysis. FIG. 21B depicts the results of a post reactive challenge PWV analysis.

FIG. 22 depicts IR thermography of two hands during a CLVR response.

FIG. 23 depicts ability of DTM to identify individuals with known CHD as compared with FRE.

FIG. 24 depicts the significant inverse linear relationships observed between DTM parameters and increasing CV risk.

FIG. 25 depicts the predictive ability of DTM and CLVR in relation to Metabolic Syndrome.

FIGS. 26A and B depict suitable designs, among others, for skin temperature sensors.

FIG. 27 depicts potential placement locations for smart Doppler arrays.

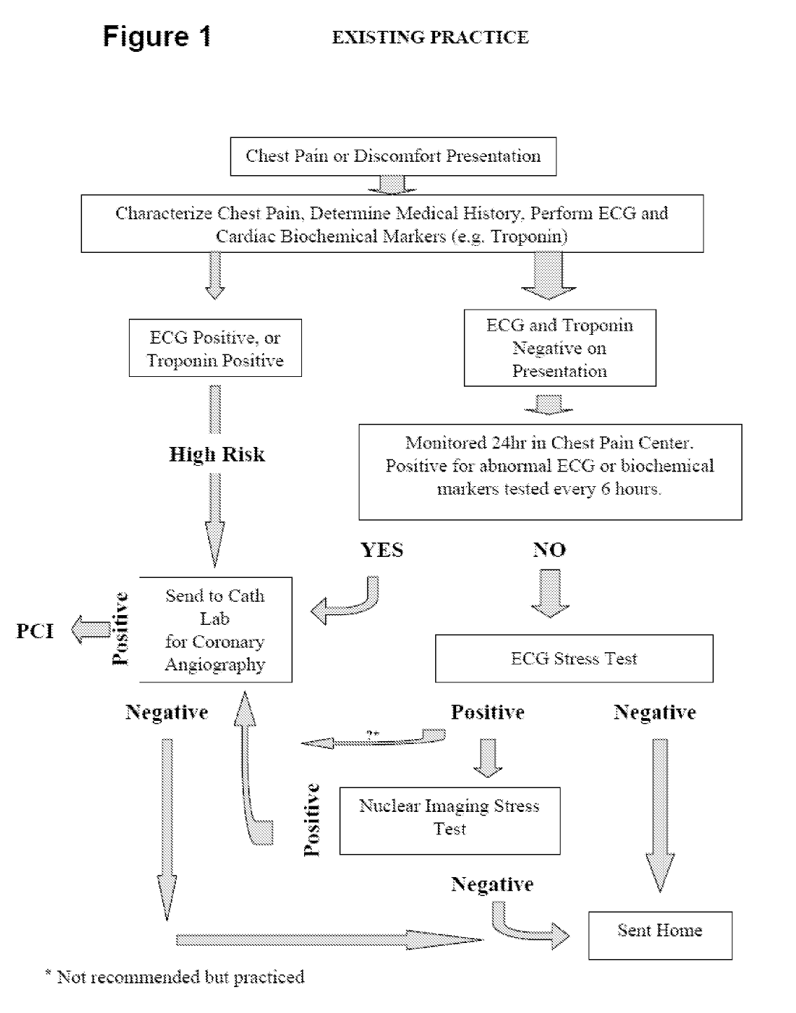

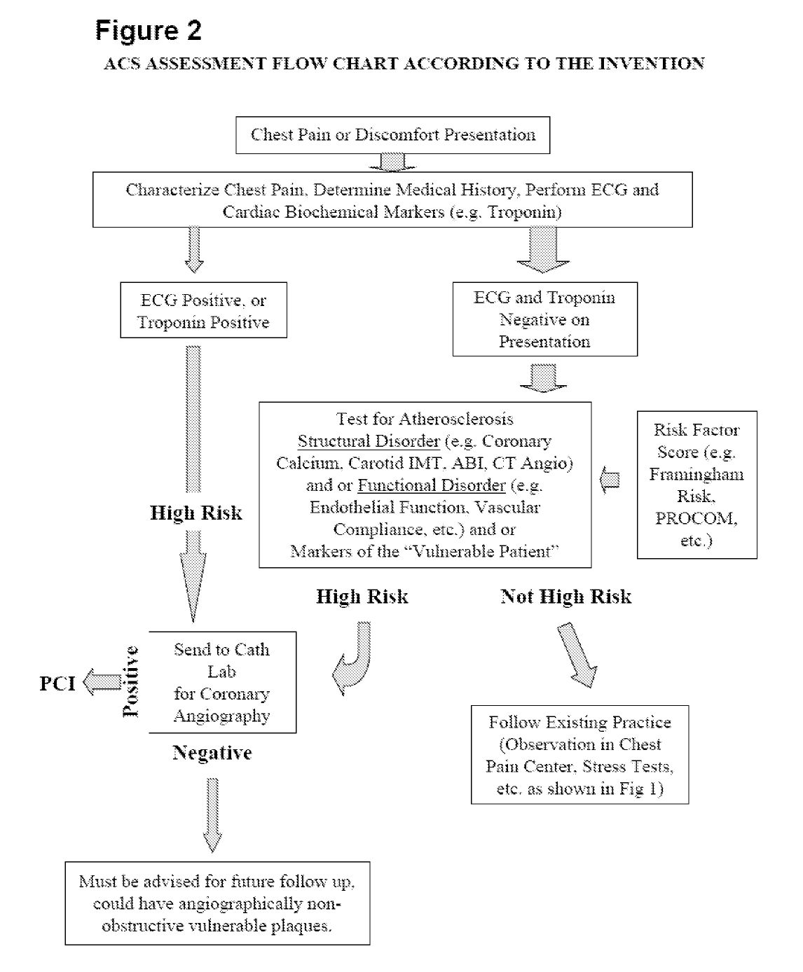

### DESCRIPTION OF THE INVENTION

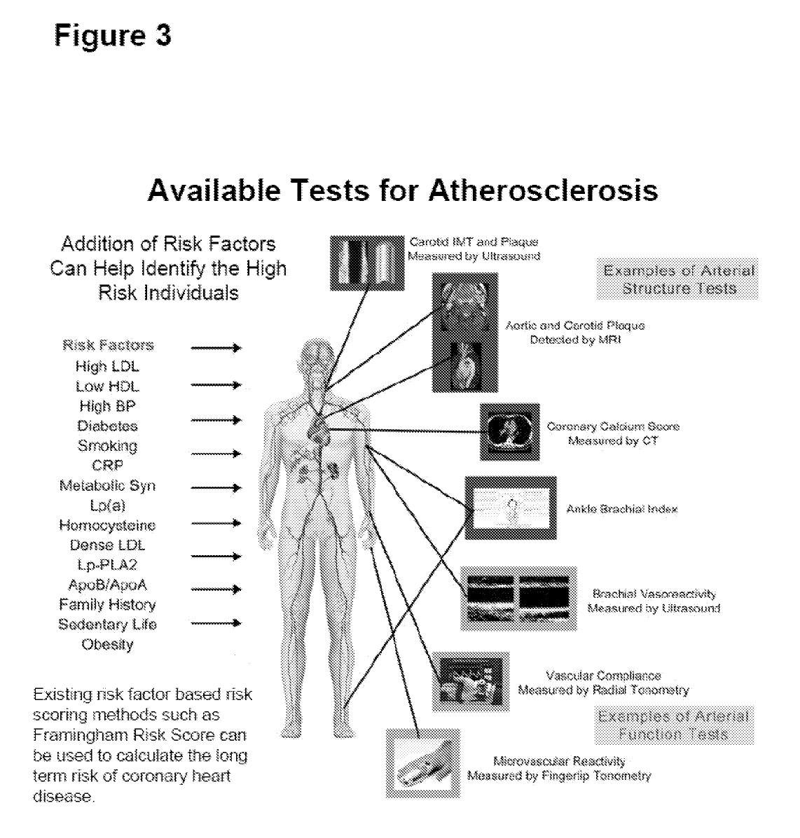

The present inventors have determined that abnormalities in vascular reactivity correlate very significantly with cardiovascular risk scoring and have applied this finding to methods and apparatus for assessing such reactivity and thus to identification of patients requiring intervention, including medical and behavioral intervention. In the present invention, determination of vascular reactivity is applied in a novel context, to acute care triage of patients presenting with chest pain. The invention is also applied to the use of other tests of vascular function or structure (e.g. tests for atherosclerosis shown in FIG. 3 below). This method will allow improvement of risk assessment in individuals suspected for acute coronary events. A combination of these tests and cardiovascular risk factors (e.g. Framingham Risk Scoring) can be used. The primary goal of this invention is to expedite the treatment of high risk individuals and minimize the unnecessary delay and associated cost in triage of these patients in chest pain centers. Also, incorporation of this additional step in the existing practice of risk assessment for chest pain may provide additional prognostic value in individuals with positive troponin test so that those with high risk result of the vascular function test who are likely to have a worse long term outcome can be subjected to receive more aggressive long term medical therapy. An example of such an additional step is CRP (C reactive protein) test which has been recently introduced to improve risk assessment of Acute Coronary Syndrome patients. Unlike troponin, CRP is not a direct and specific marker of cardiac injury, however its use in risk assessment of patients with chest pain has been argued based on its potential value in providing long term prognostic value.

In one embodiment of the invention, initial screening includes an assessment of whether or not the patient manifests demonstrable atherosclerosis as depicted for risk assessment in FIG. 3 that is not in the context of evaluation of chest pain.

The present invention thus provides a method for identifying a high risk status in an individual suspected of an acute cardiovascular event by performing a vascular function or structure test on the patient. The vascular function can be a test of vascular reactivity such as digital thermal monitoring of vascular reactivity, as described in WO04/17905 and WO05/118516 (previously invented by the inventors of this invention and incorporated herein by reference)) or other tests for assessment of vascular function such as endothelial function measurement using peripheral arterial tonometry (EndoPAT, Itamar Medical LTD, Israel), vascular compliance measurement using pulse waveform analysis (CV Profiler, Hypertension Diagnostic Inc.) or vascular stiffness using pulse wave velocity (Colin Medical/Omron Healthcare). An abnormal response on the vascular function test identifies patients in which vascular health is compromised.

The endothelium is a seminal component of the microvascular system and has many important functions in maintaining the patency and integrity of the arterial system. The endothelium regulates vascular homeostasis by elaborating a variety of paracrine factors that act locally in the blood vessel wall and lumen. Under normal conditions, these aspects of the endothelium, hereinafter referred to as “endothelial factors”, maintain normal vascular tone, blood fluidity, and limit vascular inflammation and smooth muscle cell proliferation.

When coronary risk factors are present, the endothelium may adopt a phenotype that facilitates inflammation, thrombosis, vasoconstriction, and atherosclerotic lesion formation. In human patients, the maladaptive endothelial phenotype manifests itself prior to the development of frank atherosclerosis and is associated with traditional risk factors such as hypercholesterolemia, hypertension, and diabetes mellitus. Patients that have undetected endothelial dysfunction are considered vulnerable patients for risk of an acute vascular crisis such as heart attack or stroke. The present invention provides a particularly rapid, non-invasive method for determining those patients that are most at risk for an impending myocardial infarction upon presenting to the hospital with chest pain.

In one embodiment of the present invention, vascular function testing for identification of a high risk of an acute cardiovascular event in a patient presenting with chest pain is undertaken by a vascular function test that monitors endothelial function. The endothelial function test is preferably a non-invasive non-imaging vascular function test that measures vascular response to reactive hyperemia induced by vascular occlusive challenge as the vasodilating stimulant.

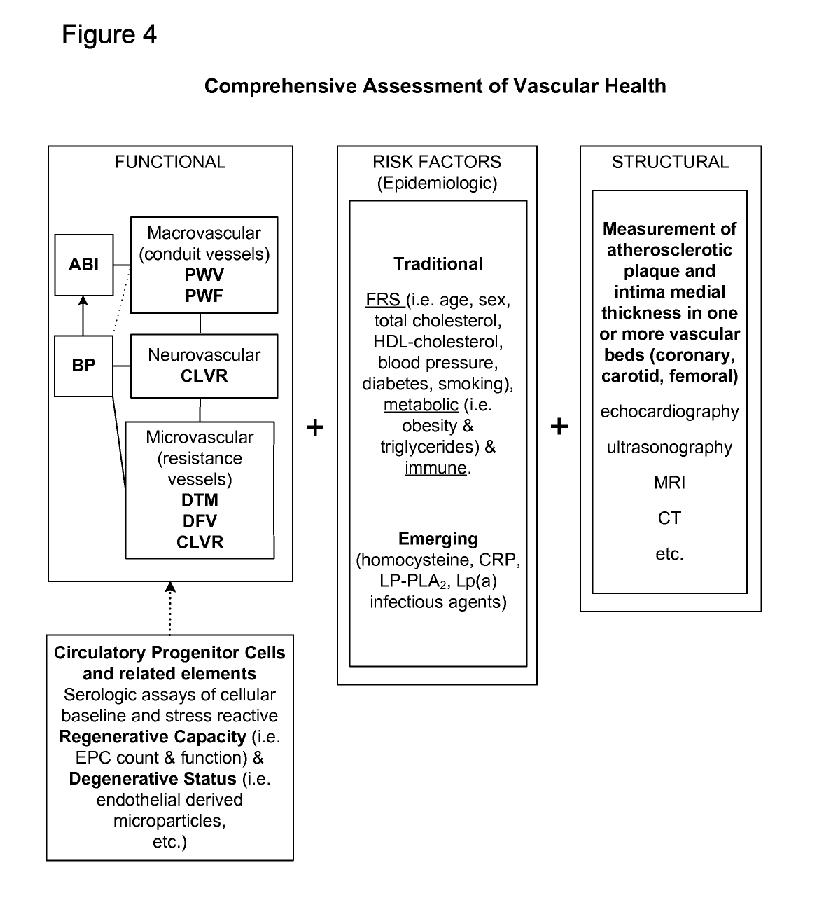

In one embodiment of the invention, methods and apparatus are provided for generating a comprehensive individual assessment of vascular health that includes functional assessments including both baseline and reactive determinations of the macrovasculature, microvasculature and neurovasculature. In further embodiments, functional assessment results are combined with inputs of risk factor assessment and structural assessment as depicted in FIG. 4 to provide a comprehensive individual determination of vascular health for purposes of determining whether an acute cardiovascular event is occurring and as a baseline for assessing future symptomatic episodes.

In accordance with one embodiment of the present invention, measurement of the functional status of both the microvasculature and the macrovasculature is provided in addition to methods and apparatus for determination of neurovascular status in the context of an acute cardiovascular event. If the functional testing indicates the presence of underlying cardiovascular disease, it is much more likely that symptomology is reflective of an existing or impending acute cardiovascular event such as a heart attack or stroke.

It is believed that the endothelial function and vascular reactivity of resistant vessels (microvasculature) can be determined by measuring changes in blood flow during a reactive hyperemia test. It is also known that changes in the diameter of non-resistant arteries subsequent to shear stress induced by increased flow reflect the endothelial function and vascular reactivity of conduit vessels (macrovasculature). Thus vascular reactivity measured during a reactive hyperemia procedure has become an established method of detecting both endothelium dependent and independent mechanisms involved in the physiologic and pathologic response to ischemia involving both the micro and macrovasculature. Vascular biology studies have shown involvement of multiple biochemical pathways in both micro and macro vascular reactivity including nitric oxide and prostaglandin pathways.

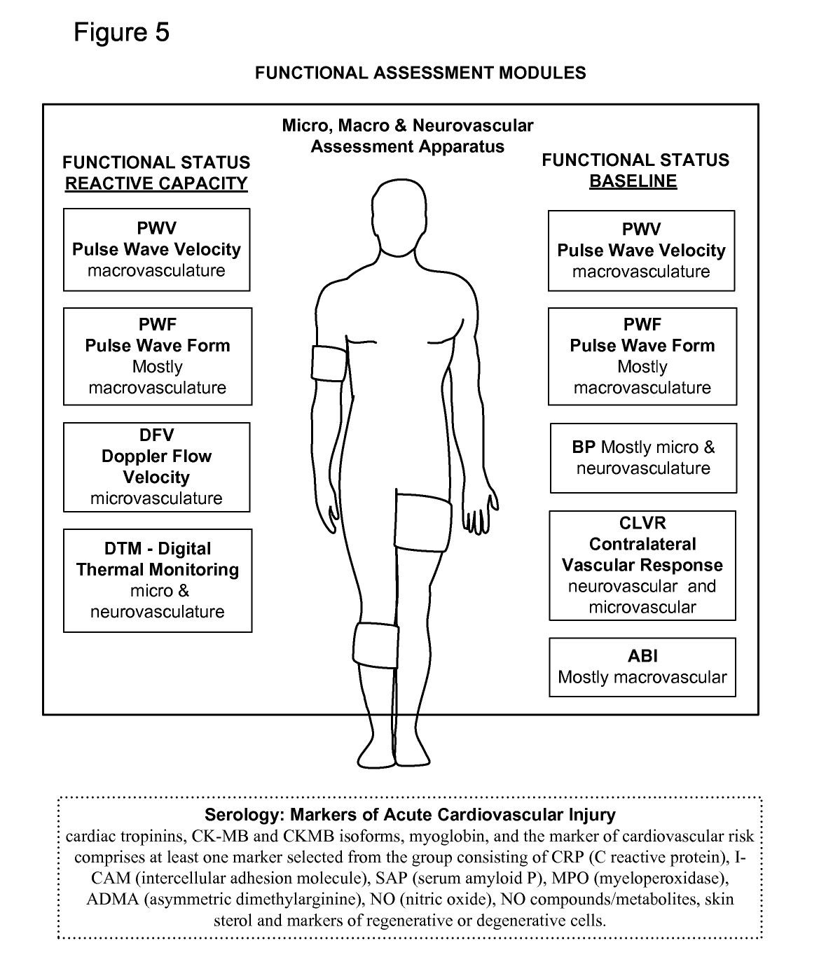

Referring now to FIG. 5, comprehensive functional assessment in accordance with the present invention includes assessment of the baseline status of the conduit vessels (macrovasculature) and the resistance vessels (microvasculature), together with neurovascular influence. The methods and apparatus provided herein can enable comprehensive assessment of the functioning of the vascular system. Assessment of the baseline and reactive status of the macrovasculature can be provided by one or more of Pulse Wave Velocity (PWV) analysis and Pulse Wave Form (PWF) analysis. Assessment of the status, both functional and structural, of the vasculature of the femoral tree can be provided by Ankle Brachial Index (ABI). Assessment of the baseline status of the combined vasculature including primarily contributions from the microvasculature and the neurovasculature is provided by blood pressure (BP) measurement. Assessment of the baseline status of the neurovascular response as combined with the ability of the microvasculature to respond is provided by measurement of the Contralateral Vascular Response (CLVR). Assessment of the baseline and reactive status of the microvasculature is be provided by Digital Thermal Monitoring (DTM) and Doppler Flow Velocity Measurement (DFV).

In one embodiment of the present invention, systems and protocols for generating a combined relative risk of underlying vascular disease are provided in accordance with FIG. 5. According to the system and method, 1) functional assessments selected from the menu of FIG. 5 are performed on an individual, 2) values obtained from the functional assessments are entered into a computational dataset for the individual, 3) results of traditional epidemiologic risk factor questioning are entered into the dataset, 3) a functional and epidemiologic risk factor combined relative risk is computed and reported for the individual. If structural data are available, this data is further added to the dataset to compute a combined comprehensive relative risk of underlying vascular disease against which presenting acute symptoms are considered. Optionally, and if structural data does not exist for the individual, and largely dependent on the functional and epidemiologic relative risk score, one or more structural assessments are performed and the data entered and computed. The risk assessment protocol described is particularly useful in assessing whether to triage the patient to the Cath-Lab.

In one embodiment, the vascular response to reactive hyperemia is determined on an extremity that is distal to a vascular occlusive challenge by vascular function measurements, at least before and after the vascular occlusive challenge, such as by inflation of a blood pressure cuff on an arm.

In one embodiment, vascular function measurements are serial temperature measurements on a finger distal to the vascular occlusive challenge. In other embodiments, the vascular response to reactive hyperemia is determined on an extremity that is distal to a vascular occlusive challenge by serial Doppler ultrasound measurements, at least before and after the vascular occlusive challenge.

In another embodiment, the vascular response to reactive hyperemia is determined on an extremity that is distal to a vascular occlusive challenge by pulse wave velocity measurements at least before and after the vascular occlusive challenge. One example of an existing instrument for conducting pulse velocity measurement is the Vascular Profiler 1000 (VP-1000) device by Colin Corporation/Omron Healthcare.

In another embodiment, the vascular response to reactive hyperemia is determined on an extremity that is distal to a vascular occlusive challenge by plethysmography. Plethysmography refers to measurement of the amplitude of a pulse wave, i.e. pulsatile finger blood flow patterns. One example of an existing instrument for conducting plethysmography on a distal extremity is the Itamar EndoPat® Device (also called peripheral arterial tonometry—PAT).

In another embodiment of the invention, vascular function test is determined by a non-invasive coronary vasoreactivity imaging test that measures change in coronary flow and/or diameter with provocation, such as by the cold-pressor test. Coronary vasoreactivity imaging can be currently performed by noninvasive echocardiography such as trans-thoracic echocardiography or by computed tomography such as non-invasive coronary CT angiography-. The transthoracic coronary echocardiography is most sensitive for assessment of left anterior descending artery (LAD).

In another embodiment of the invention, vascular function is assessed and considered in conjunction with determination of pathologic markers of cardiovascular risk, including by determination of levels of CRP (C reactive protein), I-CAM (inter-cellular adhesion molecule), SAP (serum amyloid P), MPO (myeloperoxidase), ADMA (asymmetric dimethylarginine), NO (nitric oxide), NO compounds/metabolites, and skin sterol (a.k.a. skin cholesterol). Other pathologic indices of cardiovascular risk include determining a coronary calcium score and/or measuring carotid artery intima-media thickness (IMT) by ultrasound or MRI. In certain cases the coronary calcium can be seen in a simple chest x-ray.

Triage of the Symptomatic Patient

In one embodiment of the present invention, a method is provided for identifying a high risk of an acute cardiovascular event in a patient presenting with chest pain including initially performing an EKG on the patient to determine an ST elevation. Typically a serologic test will also be performed for abnormal levels of cardiac specific biochemical markers are available including creatine kinase-MB (CK-MB) and CK-MB isoforms, myoglobin and cardiac tropinins (including cTnI and cTnT). Elevated levels of cardiac tropinins are currently the gold standard for identification of damage to the cardiac muscle. Typically, and in accordance with the current AMI assessment guidelines of the ACC, if the ST-Segment is elevated and/or cardiac specific markers are abnormal, the patient is sent to the cath lab. In this population it is unlikely that the additional vascular function test of the present invention would change the current path for immediate diagnosis of the chest pain, however, the addition of vascular testing can provide prognostic values in long term management of troponin positive individuals. In other words, those with an abnormal vascular function test (or a combination of the above) will be watched more carefully and treated more aggressive for the prevention of future adverse events and for improvement in outcome.

If the ST-Segment is not elevated and the cTn values are normal, the present invention provides that a vascular function test is performed on the patient to determine if further evaluation is required. Thus, the invention provides a new paradigm for evaluation of NSEMI patients in accordance with FIG. 2 herein. In one embodiment as depicted in FIG. 2, a further step of determining risk factor based cardiovascular risk scoring is determined for the patient and considered together with the vascular function determination. FIG. 5 depicts functional baseline and reactive tests that are envisioned and may be provided by utilizing one or more of the modular components of the apparatus disclosed herein.

One non-limiting example of what is meant by cardiovascular risk scoring is a Framingham Risk Scoring. In another embodiment, vascular function is considered together with a cardiovascular risk scoring and a coronary calcium determination. If the vascular function test, alone or particularly in combination with the risk score and the coronary calcium levels are normal, the patient is considered of low risk for an existing or impeding AMI and is worked up for other non-cardiac causes of chest pain or discomfort, including in particular gastric abnormalities such as gall stones, etc. that are not typically acute emergencies.

In one embodiment of the invention, the disclosed method of risk assessment is applied to testing and implementing therapies in clinical trials, especially for defining the inclusion criteria in clinical trials of therapeutic interventions. In one such embodiment, subjects are stratified according to the results of a non-invasive non-imaging vascular function test, for example, “normal” or “abnormal” according to predefined cut-off values. Optionally, subjects are further stratified according to the results on existing screening tests. In one embodiment, the risk assessment method is employed for establishing treatment groups for acute coronary syndrome interventions. For one ACS treatment example, subjects are assigned to the intervention as follows:

| | Vascular function | Vascular function |
|---|---|---|
| | test normal | test abnormal |
| Test result | (VF+) | (VF−) |
| Troponin test normal (TT+) | VF+/TT+ | VF−/TT+ |
| Troponin test abnormal (TT−) | VF+/TT− | VF−/TT+ |

Outcomes of the intervention compared to a control (such as placebo, or usual care) are analyzed for each of the assigned groups.

In another embodiment, treatment groups for evaluation of new treatments for cardiovascular disease are established according to the risk stratification of the invention. In another embodiment, risk stratification according to the invention is established as a population characteristic that is considered in statistical evaluation of treatment responders and non-responders at the conclusion of the trial.

In another embodiment, a similar risk assessment procedure according to the invention is made for individuals with symptoms of stroke. In the case of stroke the evaluation and management algorithms differ, but the basic approaches of risk stratification, costly imaging and observation over time is similar.

The addition of the presently described atherosclerosis tests, including vascular function, may sometimes only partially improve the risk assessment. For example, low risk individuals (e.g. troponin negative or low troponin) who show an abnormal (high risk) test result in the additional step introduced through this invention, may not be immediately routed to the high risk group (e.g. troponin positive) but may be monitored (triaged) differently. For example, such a positive test can make the medical staff in the observation room aware of the high likelihood of cardiovascular disease in such individuals which may result in better decision making based on additional borderline test results. This concept is well known among experts in light of the Bayes' Theorem and other probabilistic methods in decision making systems (e.g. neural network and many other methods known as artificial intelligence). In summary, incorporating the additional step introduced through this invention can save unnecessary cost and reduce adverse cardiovascular events.

Also, it is noteworthy that according to various studies, currently a range of 2-6% of individuals with chest pain who are discharged from hospital are high risk (due to silent MI, silent ischemia, etc) and come back with recurrent cardiac chest pain or fatal consequences. Incorporating the additional step recommended in this invention can minimize the missed high risk cased and its subsequent malpractice liabilities that confront hospitals.

Modular Micro, Macro and Neurovascular Assessment Apparatus:

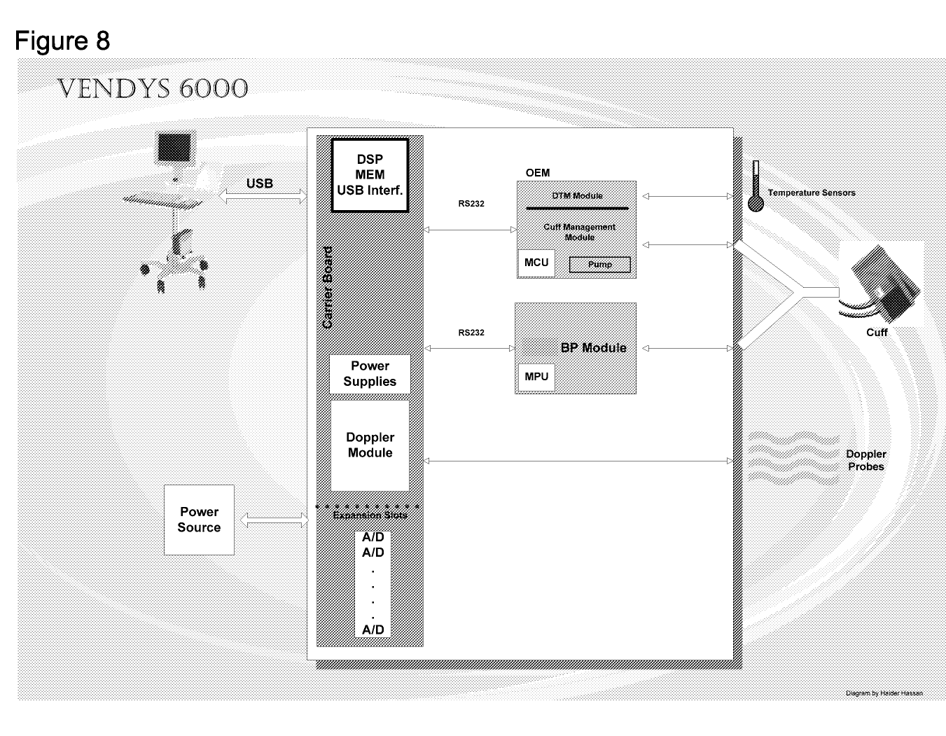

In one embodiment of the present invention a modular measurement apparatus for providing some or all of the functional assessment modules included in the Micro, Macro and Neurovasuclar Assessment Apparatus Block of FIG. 5. By providing this apparatus to health care responders such as in a hospital or clinic emergency room or to ambulance units, an underlying vascular health status for a patient can be determined for the first time. The apparatus can be customized to include one or more of the listed components, as well as further additional components. A block diagram depicting one embodiment of a basic system level design is provided in FIG. 8. In addition, apparatus will have the following features, which will be described in turn: a central processing unit (CPU) and monitor; resident GUI application residing in the CPU; a cuff management module; a DTM module; and a BP module, and will in addition include one or more of optional modules to measure PWV, PWF, DFW, and/or CLVR. In preferred embodiments the modular apparatus will include a console to house the modules and will preferably provide a compact solution for the integrated assessment modules as well as a cart to carry the CPU, monitor, and all above and mentioned components (e.g. Cuffs, Probes, etc) in addition to optional modules. In a preferred embodiment, the CPU will be interfaced with the Console, such as by USB. The monitor will preferably provide assess to the Graphical User Interface and will display graphs and data analysis in real time.

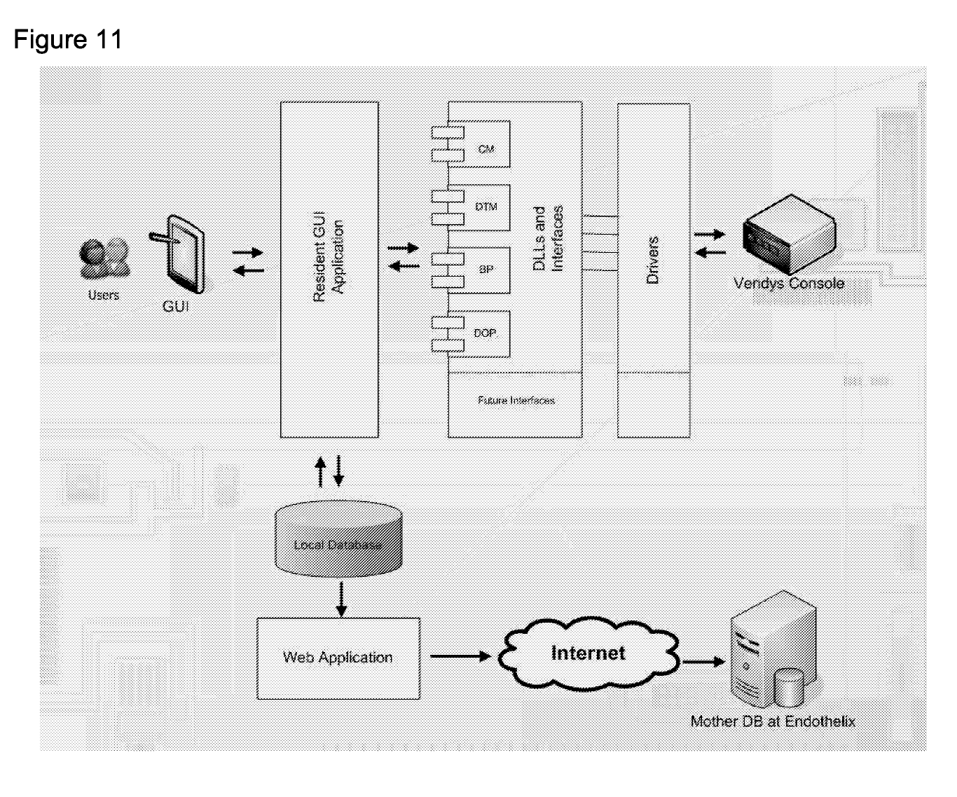

Resident GUI Application: Software will be the primary component of the device that will allow the user to use each of the modules. This software will communicate with and manage each module. Preferably it will provide the user with an attractive and easy to use Graphical User Interface (GUI) to perform the tests. This software will also direct storage of the acquired data into a local database. In one embodiment, a web component is included able to transmit the data over the internet and store it into the mother database. The Resident GUI Application (FIG. 11) will reside on the CPU. This application will communicate with each of the hardware devices through DLLs and Interfaces. This application will gather data from each device and display it on a monitor for the user. Preferably real time graphing techniques will be available. The GUI will allow the user to program certain features of the test (e.g. inflation pressure, occlusion time, etc) and to select which modules are implemented. Another purpose of this application is to store the data acquired from the modules and patient information into a local database that may reside in the same or a different CPU.

Cuff Management Module (CMM): The Modular Micro and Macrovascular Assessment Apparatus will preferably include a Cuff Occlusion Module (CMM) that will be responsible for enabling the reactive hyperemia tests using the occlusion principle. In one embodiment, occlusion will be fully automated to perform the test at an on-demand or pre-programmed basis. This module will also incorporate data reception and transmission capabilities so that remote monitoring and data gather operations are possible as depicted in the block diagram of FIG. 10.

One embodiment of the CMM will have the following features:
Ability to inflate and deflate cuffs of various sizes (e.g. arm, wrist, finger, ankle, and possibly thigh) and also manage at least two cuffs simultaneously at different pressures.
Ability to pump air quickly and will have a pressure detection mechanism.
Automated cuff inflation and deflation programmed to work for a specific time.
Safety mechanisms in case of over inflation or over duration.
Ability to accept commands of an agreed upon protocol from an external device (e.g. CPU) to carry out the specified tasks.
Ability to report any errors/malfunctions that may occur during the procedure.
Physical connector interface with the Carrier Board (CB), including preferably an ability to slide in with CBs plug and play mechanism and communicate over RS232.
Designed so as to not over heat or cause EMI.

In an alternative embodiment, the CMM comprises a plurality of cuffs, for occluding blood flow from the vessel of interest (e.g. arm, finger, ankle, etc) and adapted to measure blood pressure prior to the testing.

In one embodiment, the CMM module includes at least at least two cuffs—similar to those employed in blood pressure measurement—placed at the extremities of the patient's limb together with associated control mechanisms. The two cuffs together serve to provide occlusion in the intervening segment. The module will respond to commands from a host device. The two cuffs, say A and B, will be capable of being inflated and deflated simultaneously or independently. The occlusion pressures and duration will be programmable. Inflation will be achieved by energizing a solenoid valve which will actuate the cuff bands. At the upstream cuff A, a pressure sensor will monitor the applied pressure and regulate it using a system of micro-pumps and vent (pressure-release) valves. The downstream cuff B will sense the upstream as well as local pressures and control the applied pressure using a separate system of micro-pumps and vent valves. Micro-chip controller timers will ensure occlusion for the programmed period of time. Deflation will be achieved by simple de-energizing the solenoid.

In a preferred embodiment, system redundancy is included to eliminate single points of failure and ensure safe operation. The safety sub-system—comprising an independent system of solenoids, micro-pumps, vent valves and a micro-chip—will prevent over-pressurization or inflation beyond a certain length of time. Pressure and time thresholds will be set in firmware so that they can be overwritten by host commands. The safety sub-system must be energized in order for the primary pressurization system to function. In the event of secondary system failure, the entire occlusion system will vent to atmospheric pressure and thereby prevent occlusion. The two micro-chips will monitor each other's health, so that both systems will need to be healthy for the CMM to work.

The CMM will be controllable (hosted) by a PC or a carrier board. The host system will be responsible for providing control signals (using standard serial communication technologies) and 12 VDC power supply. During normal use, the CMM will be hosted by the carrier board, whereas during testing and firmware upgrades the PC interface will provide greater ease of use.

Digital Thermal Monitoring (DTM): Certain of the present inventors have developed novel methods and apparatus to determine the vascular reactivity based on a measured response of the vasculature to reactive hyperemia utilizing continuous skin monitoring of inherent temperature on a digit distal (downstream) to an occluded arterial flow. By inherent temperature it is meant the unmodified temperature of the skin as opposed to measurement of the dissipation of induced temperature. This principal and technique has been termed Digital Thermal Monitoring (DTM). See WO 05/18516 and U.S. patent application Ser. No. 11/563,676, the disclosures of which are incorporated herein by reference.

It is well known that tissue temperature is a direct result of blood perfusion, but other parameters also contribute. These parameters can be classified as:
Anthropometric factors, such as tissue composition, skin thickness, fat content, surface area, tissue volume, body mass index, age and gender, among others.
Environmental factors, ambient temperature, the presence of air currents, unequal radiation, air humidity and posture.
Hemodynamic factors, due to the presence of large proximal conduit arteries and small vessels and capillaries, which respond differently to occlusion and reperfusion, and have different contributions to tissue temperature.
Physiological factors, i.e. body temperature, skin temperature, tissue metabolism, response of conduit vessel diameter to hypoxia and ischemia, microvasculature response, and the activation of arteriovenous anastomoses.

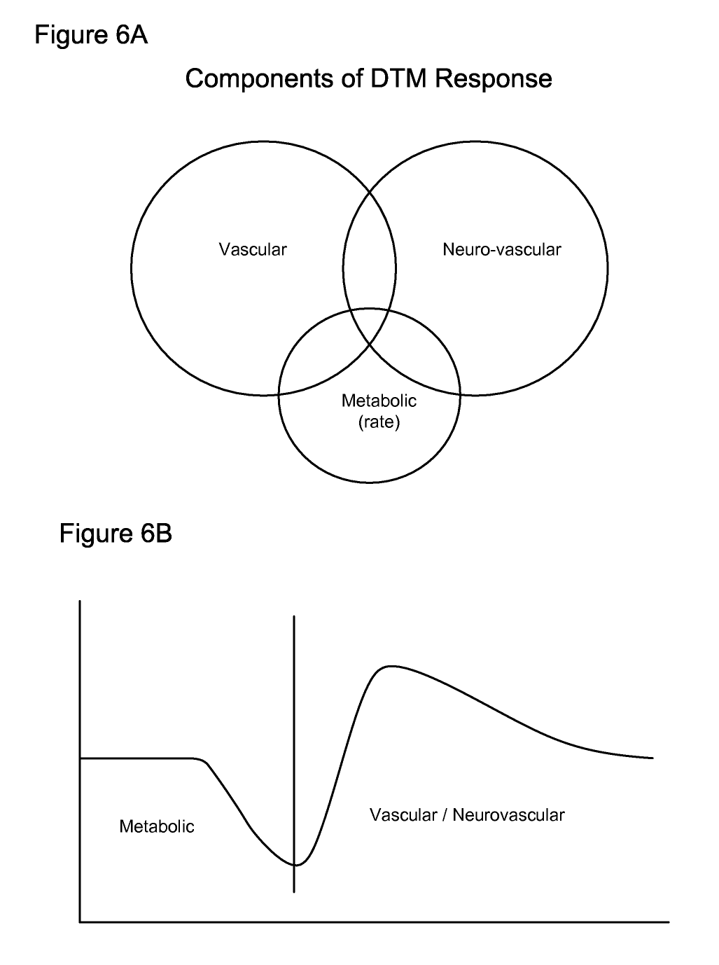

Different embodiments of this invention characterize and quantify the effect of different factors that affect the baseline temperature and temperature response observed after brachial artery occlusion. FIGS. 6A and B depict the relative combined effects of vascular, neurovascular and metabolic components to a measured DTM response.

DTM is typically implemented by measuring temperature changes at the fingertips during reactive hyperemia induced by transient arm-cuff occlusion and subsequent release. A normal reactive hyperemia response, i.e. increased blood flow after occlusion, is manifest by increased skin temperature over the baseline temperature established prior to occlusion. In an exemplary embodiment, DTM is implemented by having a subject quietly situated, such as by sitting or laying, with the forearms supported. DTM probes are affixed to the index finger of each hand. The digital thermal response during and after brachial artery occlusion is recorded and the resulting thermographs indicate temperature change during the procedure.

Since endothelial function is a systemic property, a localized measurement in a readily accessible location of the human body (such as the digits) can provide an accurate assessment of vascular health in physiologically critical locations such as the coronary arteries. DTM is thus being developed as a new surrogate for endothelial function monitoring that is non-invasive, operator-independent (observer-independent) and is sufficiently straightforward to be readily implemented across the population to assess individual vascular function. Preliminary studies have shown that digit temperature correlates significantly with brachial artery reactivity and thus provides a novel and simple method for assessing endothelial function. Further studies have shown that DTM can discriminate individuals with established CHD or high risk of future CHD (as measured by Framingham Risk Score) from normal and low-risk individuals.

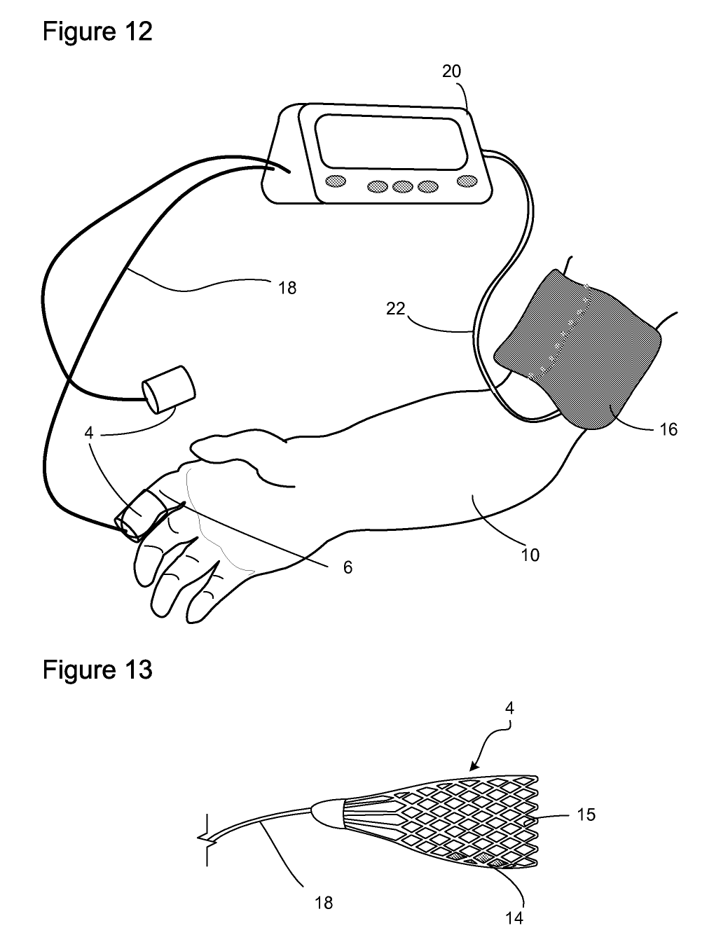

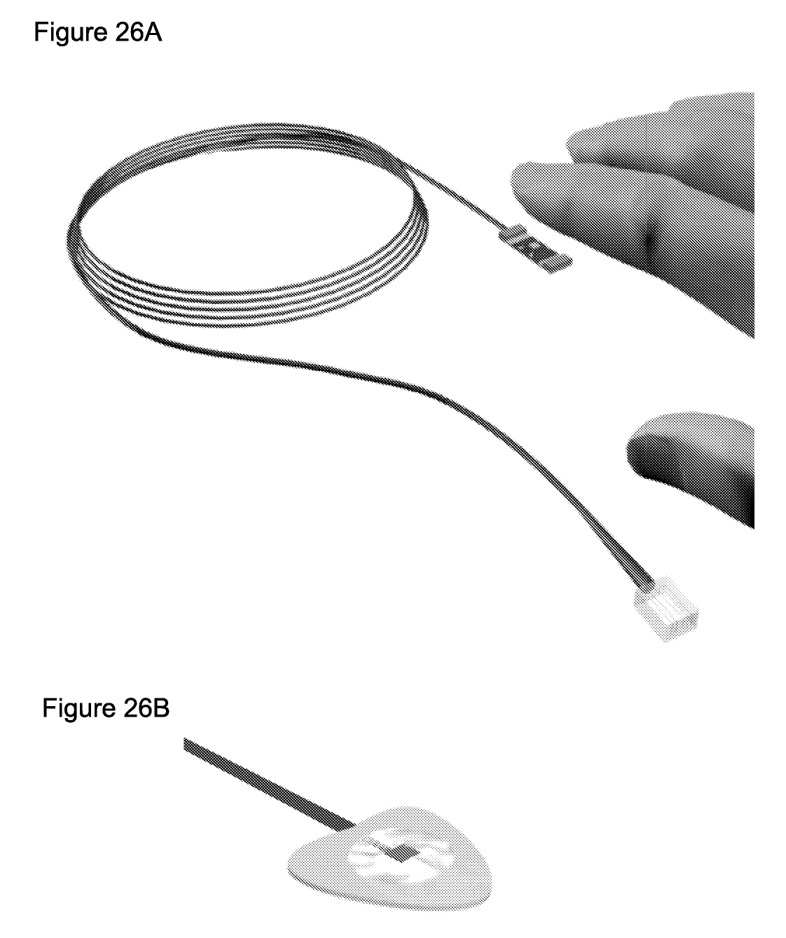

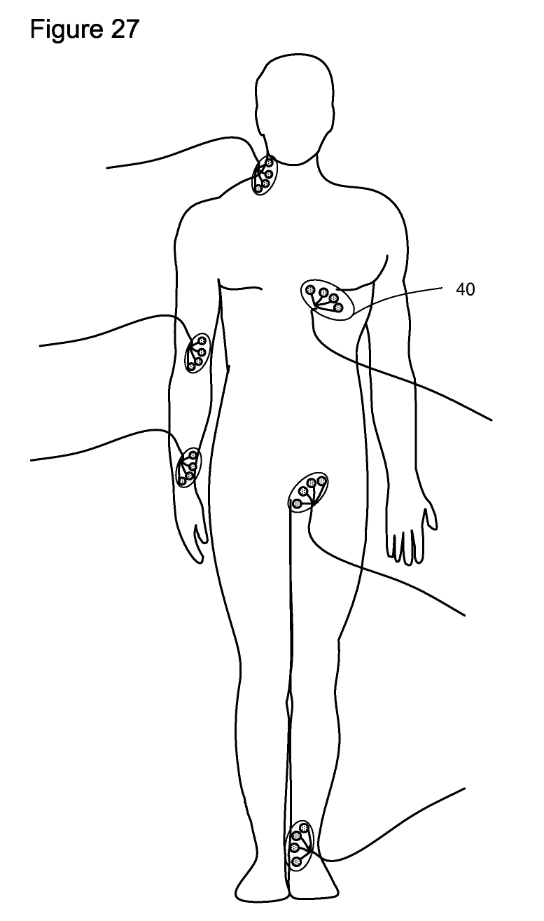

In the method, a sensitive digital thermal monitoring (DTM) device, similar to that depicted in FIG. 12, is used to measure changes in temperature at the index fingertip 6 of an arm 10 before, during and after brachial artery occlusion (200 mmHg, 2-5 minutes) using a blood pressure cuff 16. The cuff is connected to controller 20 via cable conduct 22. In one embodiment, the temperature sensor employed is a thermocouple. However, other temperature sensors might be alternatively employed in the implementation of DTM, including Resistance Temperature Detectors (RTM), thermisters, thermopiles or integrated circuit (IC) detectors. In one embodiment, as depicted in FIG. 13, the thermocouple 14 is disposed with in a basket like sleeve 15 of temperature sensor 4. In one embodiment, the temperature sensor 4 is in electrical communication via a cable 18 to the main control unit 20. FIGS. 26A and B depict suitable designs, among others, for skin temperature sensors.

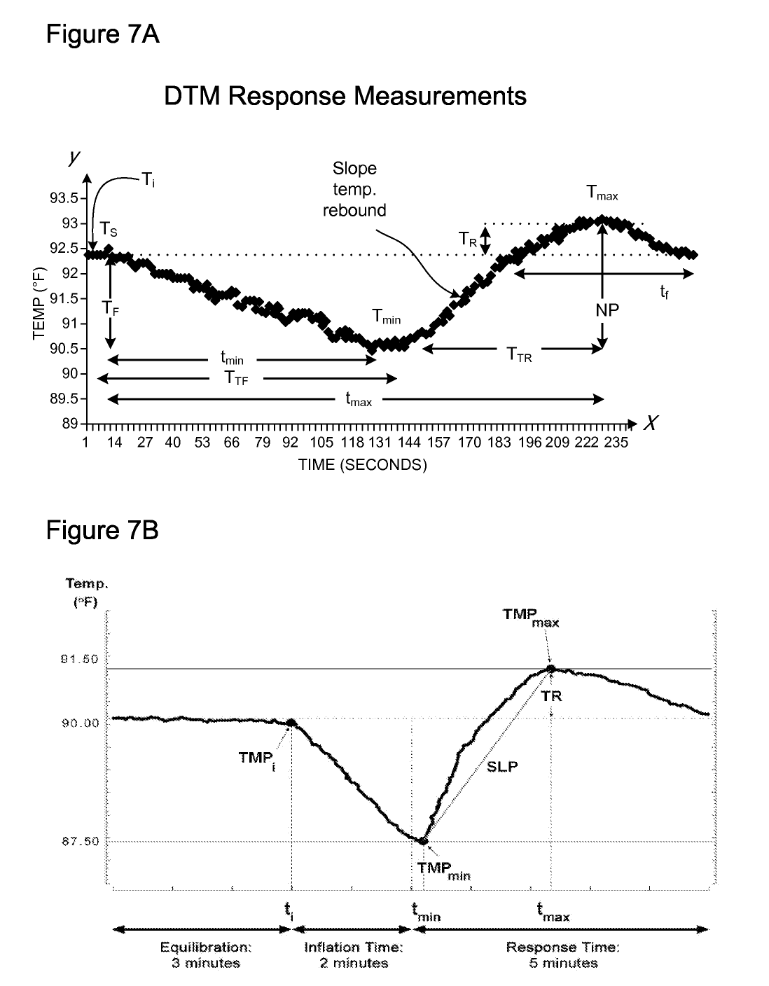

FIGS. 7A and B present actual DTM responses for the occluded hand. The following primary parameters can be calculated as depicted on FIG. 7A:

Measures reflecting the ischemic stimulus/thermal debt:

| | |
|---|---|
| T_{S} | Starting fingertip temperature |
| Tₘᵢₙ | (Nadir (N)) Lowest temperature observed after cuff inflation |
| T_{F} | Temperature Fall, T_{S} − Tₘᵢₙ |
| T_{TF} | Time from cuff release to Tf (tₘᵢₙ₋tᵢ) |
| tᵢ | Time when the initial temperature was recorded |
| tₘᵢₙ | Time taken to attain Tₘᵢₙ |
| tₘₐₓ | Time to attain maximum temperature |
| t_{f} | Time to attain the equilibrium temperature (final temperature). |

Parameters reflecting thermal recovery/vascular reactivity:

| | |
|---|---|
| Tₘₐₓ | Highest temperature observed after cuff deflation |
| T_{R} | Tₘₐₓ − T_{S} (temperature recovery/rebound) |
| NP | Nadir-to-Peak, Tₘₐₓ − Tₘᵢₙ |
| T_{TR} | Time from cuff release to T_{R}, (tₘₐₓ₋tₘᵢₙ) |
| SlopeT_{R} | Slope of temperature recovery = NP/(T_{TR}) |
| AUC | Area under the temperature-time curve |

T_{R} and NP indicate the vasodilatory capacity of the vascular bed (small arteries and micro-vessels) and subsequent hyperemia induced brachial artery dilation. T_{R} specifically denotes the ability of the arterial bed to compensate for the duration of the ischemia and to create an overflow (hyperemia) above the baseline level. Given a good vasodilatory response and constant room temperature one would expect a positive T_{R}. The higher the T_{R}, the higher the vasodilatory response of the arterial bed. T_{R} close to zero indicates a lack of strong vasodilatory response and negative T_{R} is likely to represent a vasoconstrictive response. NP and T_{R} largely overlap and both show similar trends with T_{R} being a more sensitive marker of overflow (hyperemia response) and NP showing additional factors that affect T_{F} (such as neuroregulatory effect and basal metabolic rate). Factors as T_{TF}, T_{TR} and area under the curve are expected to provide additional insights into the response to the ischemia challenge test.

A simplified set of DTM values can be utilized as depicted in FIG. 7B and as defined below. Although different terminology may be employed between FIGS. 7A and 7B the critically measured components are essentially the same:

Temperature (T)

| | |
|---|---|
| TMPi | Initial fingertip temperature at cuff inflation |
| TMPₘᵢₙ | Lowest temperature (nadir) observed after cuff |
| | inflation |
| TMPₘₐₓ | Highest temperature observed after cuff deflation |

Time (t)

| | |
|---|---|
| tᵢ | Time of cuff inflation |
| tₘᵢₙ | Time of TMPₘᵢₙ |
| tₘₐₓ | Time of TMPₘₐₓ |

Derived Parameters

| | |
|---|---|
| TR | Temperature Fall = TMPᵢ − TMPₘᵢₙ |
| TF | Temperature Rebound (TMPₘₐₓ − TMPᵢ) |
| NP | Nadir to Peak (TMPₘₐₓ − TMPₘᵢₙ) |
| SLP | Slope (TMPₘₐₓ − TMPₘᵢₙ)/TMPᵢ) |

Normalized Derived Parameters

| | |
|---|---|
| TMPₘₐₓ % | (TMPₘₐₓ/TMPᵢ) × 100 |
| NP % | ((TMPₘₐₓ − TMPₘᵢₙ)/TMPᵢ)) × 100 |
| SLP % | ((TMPₘₐₓ − TMPₘᵢₙ) × (tₘₐₓ − tₘᵢₙ)) × 100 |

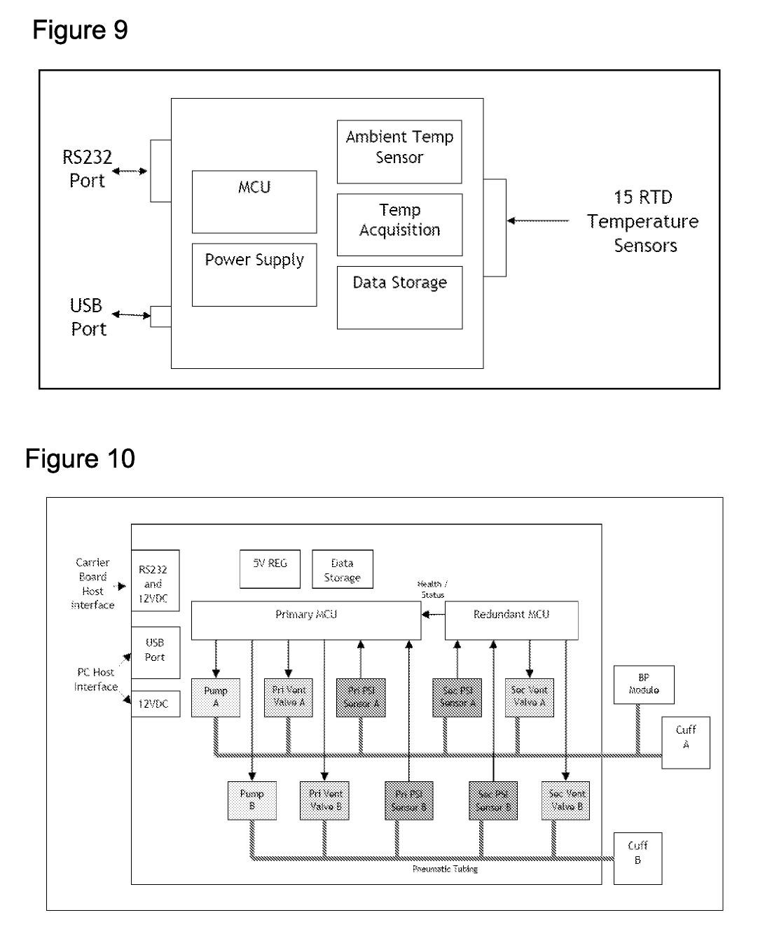

In one embodiment, the DTM module controller (FIG. 9) will be an analog data acquisition printed circuit board (PCB). It will be used in DTM testing to monitor temperature changes in the fingers due to blood occlusion. It will be interfaced with the temperature probes. It will gather temperature data, convert it into a digital format and transmit it to an external device. This module is designed to perform various functions including the following:
Capability for data acquisition from multiple RTD temperature probes.
Data conversion into a datagram of an agreed upon protocol to the external devices and also perform data transmission via RS232 protocol.
Uses minimal power and will not overheat and cause EMI.
Easy installation and adequate software support to make interfacing with the CPU staightforward.
Designed to report errors/malfunctions that may occur during the procedure.

In a preferred embodiment, the DTM comprises a main control unit (MCU), a power supply for the temperature sensors (RTDs), an ambient temperature sensor, a temperature acquisition unit and a data storage unit. The entire module is controlled by a host device, either be a PC or a carrier board. The host can communicate with the module using standard serial communication technologies.

Control will be achieved using a well defined set of commands, such as initialize, get temperature, reset, calibrate, etc. Upon receiving an initiate command, the data acquisition unit reads temperatures from a plurality of RTD sensors. A large number of sensors may be used to attain a high signal-to-noise ratio using filtering and averaging techniques. The DTM constantly monitors and filters the temperature readings from all the sensors. To retrieve the measurement, the host is expected to send read commands at a fixed frequency for the duration of the test; a faster internal sampling frequency will be employed to ensure adequate data for filtering purposes. In one embodiment, the DTM returns an 8-bit status code indicating the health of the device and the measurements. In a preferred embodiment, to further attain high accuracy sensor self-heating will be limited by applying a sensor voltage bias to each sensor for a short duty cycle. In one embodiment a boot-loader mechanism is be provided to enable new versions of firmware to be installed via the PC interface mechanism.

In one embodiment of the invention, changes in skin temperature before, during, and after an ischemia challenge are measured and related to the underlying vascular, metabolic, and neuroregulatory functions of the tissues. In one embodiment, repeated measurement of the temperature response as well as testing temperature responses in multiple vascular beds including the arm, forearm, wrist, and both legs provides a more comprehensive assessment. For example, the AV shunts in digital capillaries can affect distal microvessel resistance and therefore the flow measurement or response to ischemic challenge can vary depending on the opening of these AV shunts as a consequence of sympathetic drive. One way to measure the AV shunt effect is to simultaneously measure temperature at the distal finger tips as well as proximal to the finger tip such as on the wrist or forearm. By comparing temperature changes in these two locations, one can create a differential signature plot that indicates the activity of the sympathetic nervous system and/or AV shunting. The modular design of the present apparatus is able to monitor and control a plurality of skin temperature measurement devices.

Blood Pressure Measurement: In an exemplary embodiment, the Modular Micro and Macrovascular Assessment Apparatus includes a module for measuring and recording the blood pressure of the subject. DTM and BP measurement are facilitated by an integrated device that provides monitoring of blood pressure in conjunction with a pressure cuff used to provide vascular occlusion as part of a DTM measurement. In one embodiment, the blood pressure of the subject is measured using Korotkoff sounds or oscillometric methods. In an alternate embodiment, blood pressure measurement is implemented by measuring radial artery waveforms to calculate systolic, diastolic and mean pressures. In alternative embodiments, the blood pressure of the subject is measured using fingertip blood pressure, wrist blood pressure. The blood pressure of the subject can be conveniently measured at one or more times including before, during, and after the provision of the vasostimulant.

The combination of BP and DTM is particularly suitable for the management of hypertension. Using different ischemia challenge protocols, one can distinguish between different stages of hypertensive vascular disease. Subjects in later stages of the disease whose vasodilatory capacity is severely reduced may show lower T_{R}. Longer duration of ischemia may distinguish this group with the earlier stages of hypertension where the vasodilatory capacity is relatively high.

Blood pressure measurement, which can be subject to high variability and White Coat effect, has evolved over time into ambulatory monitoring including use outside of the hospital. Similarly, measurement of brachial vasoreactivity, including as measured by DTM, may show marked variations including diurnal, postprandial, positional, exercise and stress related variability. Solutions to control for variability issues include multiple measurements and standardized settings for measurement. A requirement for multiple measurements cannot be met by BAUS, which is a very complicated, cumbersome and expensive measurement. In contrast, DTM has great potential to provide an endothelial function measurement device capable of ambulatory monitoring. Such a device, including combined with blood pressure monitoring device, can provide an excellent tool for screening and monitoring of vascular function at minimum cost.

Ankle Brachial Index (ABI) Module: In one embodiment of the invention, a module is provided for ankle brachial index (ABI) determination. ABI is a useful test to assess lower extremity arterial perfusion. The ABI is particularly useful in define the severity of Peripheral Vascular Disease (PVD), also known as peripheral arterial disease (PAD). (PVD) affects more than 8-10 million Americans and is a risk marker for coronary disease, cerebrovascular disease, aneurysmal disease, diabetes, hypertension, and many other conditions. Indeed patients with documented PVD have a four- to six-fold increase in cardiovascular mortality rate over healthy age-matched individuals. However, fifty percent of people with PVD are symptomatic.

The Modular Apparatus of the present invention is adaptable for ABI determination. Flow detection for determination of the ABI is traditionally performed using continuous wave Doppler. Thus, one of more of the Doppler sensors of the Modular Apparatus can be utilized to determine blood pressure at the brachial artery and over the ankle. The two values are compared by the unit's software and an ABI index is calculated and reported. Although Doppler is typically utilized for detecting resumption of flow as occlusion pressure is gradually released over the arm and ankle, other means may be suitable such as the reported use of photoplethysmography (PPG) sensors for flow detection (B. Jönsson, et al. A New Probe for Ankle Systolic Pressure Measurement Using Photoplethysmography (PPG). Annals of Biomedical Engineering 33:2, 232 (2005)).

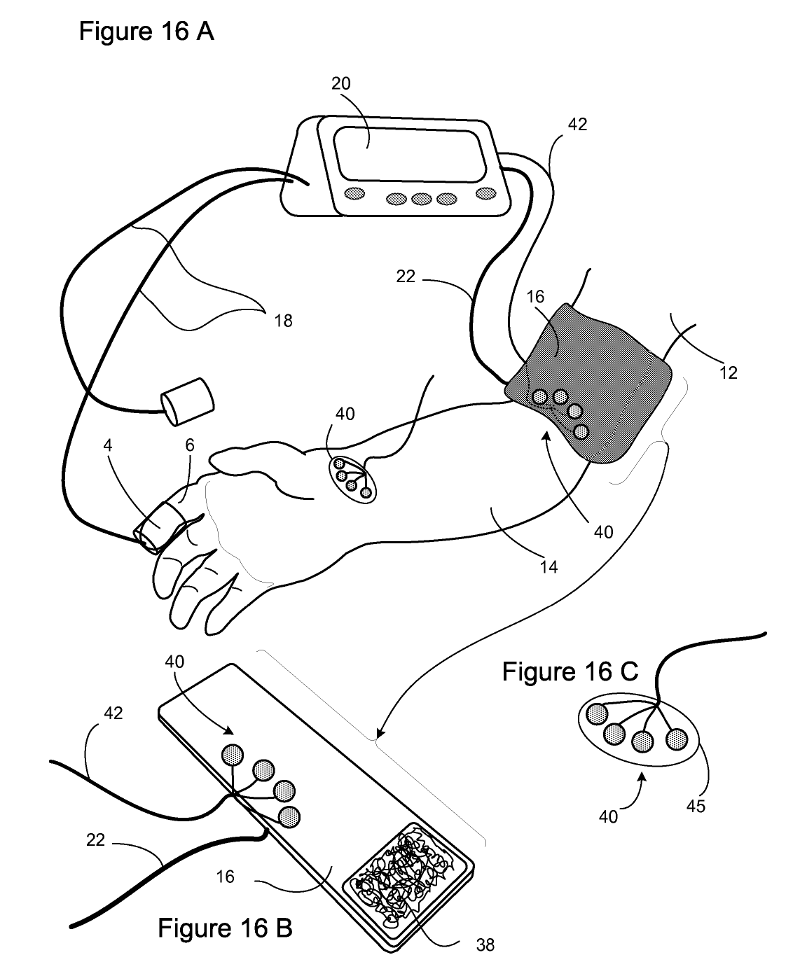

In one embodiment of the invention, a combined blood pressure cuff 16 and flow sensor array 40 is utilized wherein the flow sensor array disposed on the inside of the cuff, such as that depicted in FIG. 16B is provided that utilizes smart technology to select the particular flow probe that gives the highest signal in the given individual. In one embodiment, the sensors are disposed in a local array. In another embodiment the sensors are placed circumferentially around the cuff. The cuff including integrated sensor array can be used at either the elbow or ankle to eliminate the variable of requiring the operator to move the flow sensor probe to the best location on the patient. In an alternative embodiment a separate sensor array such as that depicted in FIG. 16C is utilized. The flow sensors are disposed in an array on patch, disk or pad 45. The patch can be self adhesive, manually held in place, or can further include a strap that goes circumferentially around the limb and can be held with a closure such as for example a Velcro type closure 38. In an alternative embodiment, the sensors are disposed in an essentially linear array that can be affixed around the arm or ankle like a strap. In one embodiment of the invention, the sensors are Doppler sensors. In another embodiment of the invention the sensors are infrared photoplethysmography sensors.

Contralateral Vascular Response (CLVR): Importantly, the present inventors have found that significant temperature changes in control arms were found in some individuals that are thought to reflect the neuroregulatory response to the cuff inflation and deflation. Thus, in one embodiment, measurements on the contralateral hand to that receiving a vascular challenge are used to establish a vascular, metabolic, and neuroregulatory profile for the patient. The present inventors have surprisingly found that, rather than being considered as “noise” to be discounted or controlled, in certain embodiments of the present invention, measurement of skin temperature on the contralateral hand is utilized to provide important insights into the vascular reactivity profile of the individual.

In contrast to the test hand to which a vascular challenge is applied, for example by occlusion of the brachial artery feeding the test hand, the contralateral hand is also monitored for blood flow changes such as by a fingertip temperature measurement on the corresponding digit of the contralateral hand but without vascular challenge to the vasculature feeding the contralateral hand. Since 85% of skin circulation is thermoregulatory and tightly controlled by the sympathetic system, changes in the contralateral finger temperature can be quite diagnostic. In some individuals the temperature of contralateral fingers goes up in the inflation phase while in other individuals the temperature of the contralateral finger declines in the deflation phase. In some patients, the contralateral finger temperature goes up in the inflation phase and declines in the deflation phase. The contralateral finger response reflects both the activity of the sympathetic nervous system but also the ability of both the nervous system and the vasculature to work together to respond appropriately to vascular challenge.

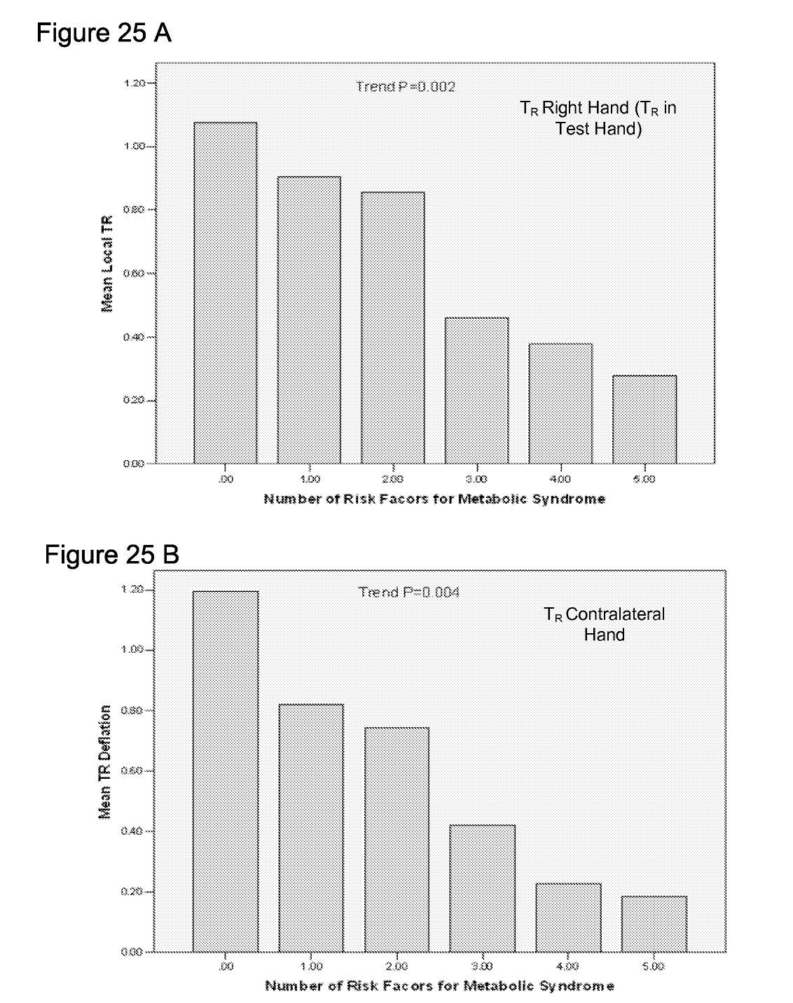

Contralateral vasomotion is believed to show the neurogenic factors involved in the arm-cuff based vascular reactivity test and provides, for the first time, the ability to provide characterization of this influence in different individuals. FIGS. 25A and 25B present a comparison of the results of correlation between the DTM T_{R} values with numbers of risk factors for metabolic syndrome in the right test hand versus the contralateral hand. FIG. 25A depicts the strong correlation between risk factors for metabolic syndrome and DTM T_{R} in the fingers of the arm that undergoes reactive hyperemic challenge. Remarkably, FIG. 25B depicts an also very strong correlation between risk factors for metabolic syndrome and DTM T_{R} values for the left contralateral hand that is not directly challenged but instead reacts on the basis of neurovascular instruction.

Physiologic stimuli such as local pain, pressure, and ischemia are known to create systemic effects mostly mediated by autonomic (sympathic and parasympathic) nervous system. DTM provides a mechanism to correlate primary and secondary autonomic disorders shown by heart rate variability, and orthostatic hypo and hyper-tension in coronary heart disease and a host of other disorders, with the thermal behavior of contralateral finger.

In one embodiment, the body part is a first hand on the subject, and the contralateral body part is a second hand on the subject. In other embodiments, the body part is a first foot on the subject, and the contralateral body part is a second foot on the subject. In an exemplary embodiment, the body part is a finger on the subject, and the contralateral body part is a toe on the subject.

Changes in blood flow in a contralateral body part as a consequence of a vascular stimulus on a corresponding test body part can be detected by temperature sensing instrumentalities including for example with a thermocouple, thermister, resistance temperature detector, heat flux detector, liquid crystal sensor, thermopile, or an infrared sensor. However, changes in blood flow in a contralateral body part as a consequence of a vascular stimulus on a corresponding test body part are not limited to temperature detection but may also be detected by skin color, nail capilloroscopy, fingertip plethysmography, oxygen saturation change, laser Doppler, near-infrared spectroscopy measurement, wash-out of induced skin temperature, and peripheral arterial tonometry.

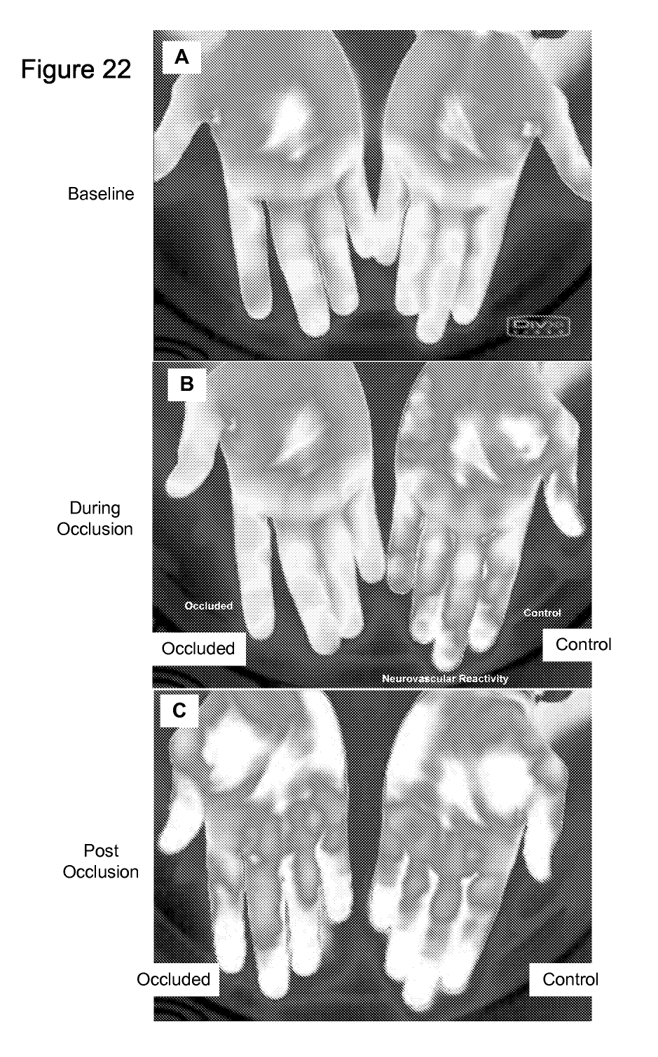

In an alternative embodiment, vascular responses in the contralateral body part are detected by infrared thermal energy measuring devices such as, for example, infrared cameras. Temperatures before, during, and after vasostimulation, such as may be provided by cuff occlusion, are measured by infrared camera. Infrared (IR) thermography is employed to study vascular health before, during, and after a direct vascular stimulant such as nitrate or cuff occlusion. For example, infrared imaging of both hands or feet during cuff occlusion test (before cuff occlusion, during and post occlusion) using infrared thermography results in a comprehensive vascular and neurovascular assessment of vascular response in both hands or feet. FIG. 22 depicts the results of IR thermography of two hands of the same individual before (A), during (B) and after (C) occlusion of the brachial artery by an inflated blood pressure cuff on the individual's right arm. In this application, quantitative measurements of temperature changes are generated by numerical analysis of each depth of color in the image. The technique typically utilizes a color map of the thermal image as shown in FIG. 22.

Pulse Wave Velocity (PWV) Module: PWV is a function vascular stiffness & dimensions and because it is modulated by compliance, PWV can be used to assess macrovascular function. PWV is typically defined mathematically as PWV2=Eh/dp, where E is Young's modulus, h is thickness, d is diameter, and p is blood density. Pulse wave velocity measurements utilize spaced apart detectors that essentially compare the time of arrival of a pulse between the spaced apart detectors. PWV can be detected by tonometry, ultrasonography, and oscillometrics, In one embodiment of the invention PWV is determined by Doppler measurements at two spaced apart sited on a single arterial tree. In one embodiment the spaced apart sites are located essentially at brachial and radial sites to detect changes in PWV in response to increased blood flow induced by reactive hyperemia (similar to FMD).

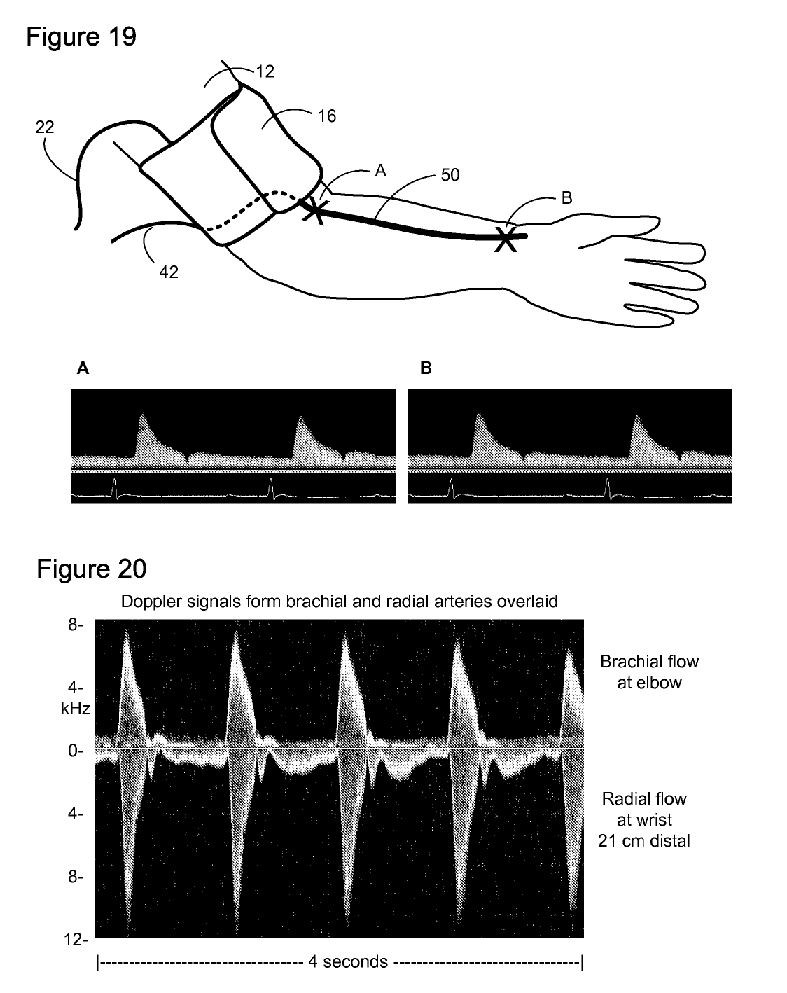

A set up for measuring pulse wave velocity is depicted in FIG. 19. As depicted, measurement of pulse wave velocity requires two probes spaced apart, such as one at point A and one at point B. In one embodiment of the invention, a template or guide 50 is provided establishing the distance between point A and point B and the placement of the probes. In one embodiment of the invention, the template or guide is a bar on which the probes are slidably mounted. In one embodiment wherein the PWV measurements are implemented using Doppler, the Doppler probes are connected to a Doppler control module via connection 42. The speed at which a pulse travels from elbow (brachial artery-point A) to wrist (radial artery-point B) can be reliably measured by simultaneous monitoring of pulse arrival time using two Doppler probes at points A and B via the CPU which is programmed to perform pulse wave velocity analysis.

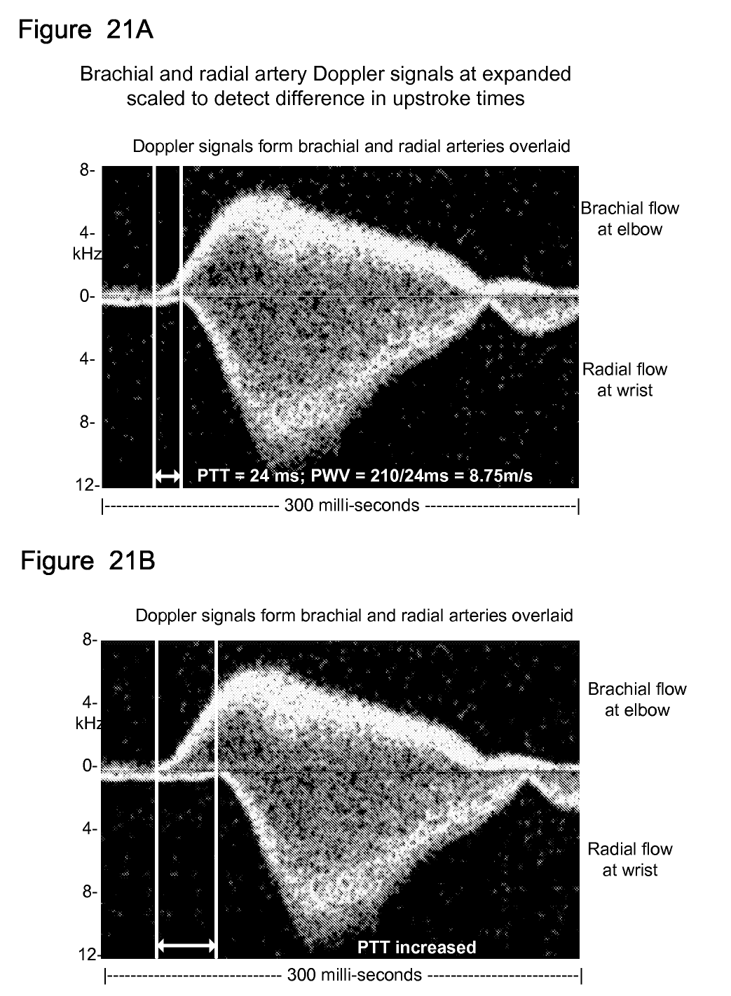

With a healthy vascular response, the pulse travel time from A to B increases after cuff deflation (indicating the intermediate artery dilatation and slowed pulse wave velocity). Analysis of the data recorded at point A and point B is overlaid as depicted in FIG. 20. By dissecting and scaling the overlays of each pulse, differences in the arrival of a single pulse from point A to Point B can be accessed by measuring the differences in upstroke times as shown in FIG. 20. FIG. 21A depicts the resulting expanded scale that permits measurement of the pulse transit time (PTT) and the derived pulse wave velocity (PWV) as a baseline measurement.

Pulse Wave Velocity can also be used to determine vascular function in response to reactive challenge. Reactive hyperemia is defined as hyperemia, or an increase in the quantity of blood flow to a body part, resulting from the restoration of its temporarily blocked blood flow. When blood flow is temporarily blocked, tissue downstream to the blockage becomes ischemic. Ischemia refers to a shortage of blood supply, and thus oxygen, to a tissue. When flow is restored, the endothelium lining the previously ischemic vasculature is subject to a large, transient shear stress. In partial response to the shear stress, the endothelium normally mediates a vasodilatory response known as flow-mediated dilatation (FMD). The vasodilatory response to shear stress is mediated by several vasodilators released by the endothelium, including nitric oxide (NO), prostaglandins (PGI₂) and endothelium-derived hyperpolarizing factor (EDHF), among others. A small FMD response is interpreted as indicating endothelial dysfunction and an associated increased risk of vascular disease or cardiac events. See Pyke K E and Tschakovsky M E “The relationship between shear stress and flow-mediated dilatation: implications for the assessment of endothelial function” J Physiol 568(2) (2005) 357-9.

Induction of reactive hyperemia is well-established in clinical research as a means to evaluate vascular health and in particular endothelial function. Typically, a reactive hyperemia procedure is implemented by occluding arterial blood flow briefly (2-5 minutes, depending on the specific protocol) in the arm, by supra-systolic inflation of a standard sphygmomanometer cuff, then releasing it rapidly to stimulate an increase in blood flow to the arm and hand. Reactive hyperemia has been classically measured by high-resolution ultrasound imaging of the brachial artery during and after arm-cuff occlusion. However, the technical difficulties of ultrasound imaging have limited the use of this test to research laboratories. This method is clearly unsuitable to widespread adoption of reactive hyperemia as a test of vascular function. The method is simply inapplicable to evaluation of endothelial function in the context of real life stress inducers.

The present inventors have adapted PWV as a more accessible measurement of FMD using Doppler detection. A baseline PWV measurement is obtained as described above. The procedure is repeated after inflation of a blood pressure cuff for sufficient time to normally induce FMD, followed by release of the cuff and immediate determination of PWV. FIG. 21B depicts an expanded scale measurement of the pulse transit time (PTT) and the derived pulse wave velocity (PWV) after release of a blood pressure cuff as compared to the baseline reading of FIG. 21A. In a healthy vasculature that is pliable and properly responsive to both ischemia and FMD, the artery is distended resulting in a measurable decrease in PTT and PWV.

In an alternative embodiment, pulse wave velocity is determined not from the velocity of natural pulses but from the velocity of an artificial pulse induced by external distal arterial tapping to create a tapped reverse wave such as described by Maltz J S and Budinger T F. WO2005/079189.

Pulse Wave Form (PWF): Arterial circulation is hemodynamically controlled by the relationship between pulsatile cardiac output and total peripheral resistance, which is modulated by vascular tone, capillary density and the wall thickness to lumen ratio in the media of the microvasculature. To the extent that they are able, the arteriolar and capillary beds provide variable resistance to flow and thereby regulate blood flow to meet the need of the tissues. PWF analysis provides a measure of the stiffness of an artery supplying blood to the body part.

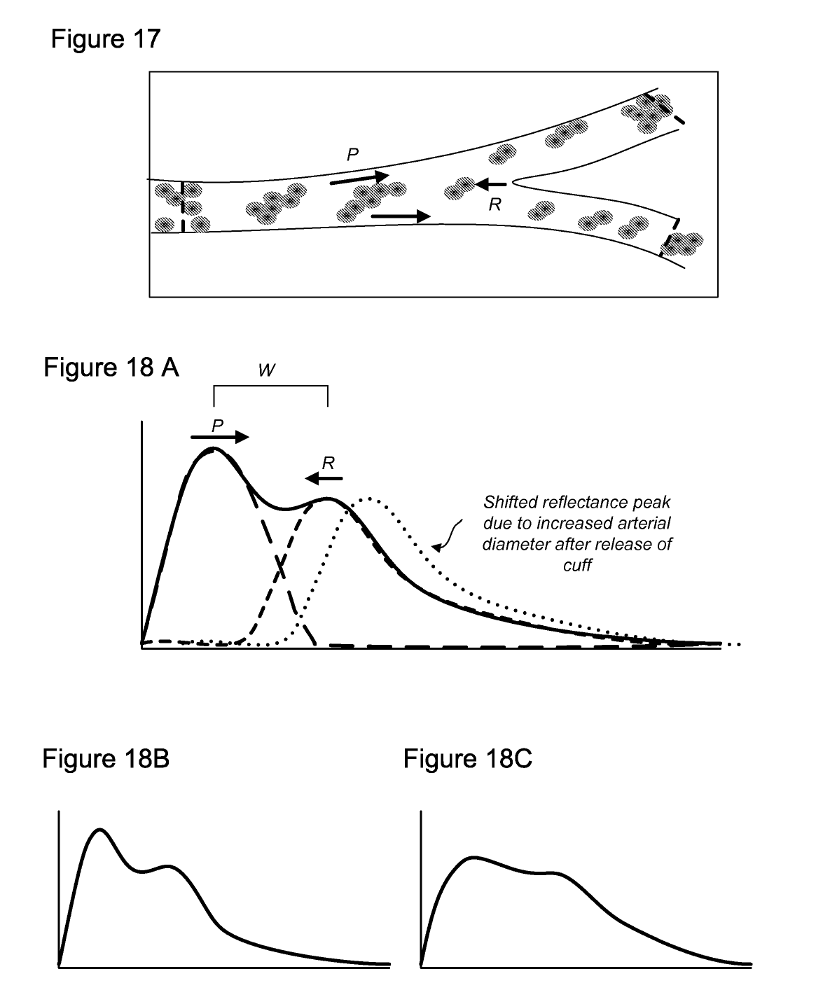

As depicted in FIG. 17, as each pulse wave, P, passes through an artery, it is met by a smaller deflected or reflectance (backward) wave, R, thus producing an oscillatory waveform as depicted in FIG. 18. The speed of travel for each pulse wave (both forward and backward) is inversely proportionate to the diameter of the artery. Analysis of the shape of a pulse valve is termed pulse wave form (PWF) or Pulse-contour analysis. Loss of the normal oscillatory waveform is believed to represent an early and sensitive marker of altered structural tone with aging and cardiovascular disease states.

Typically pulse wave form analysis is determined by use of a single Doppler probe. If there is an increase in the diameter of the artery (e.g. induced by reactive hyperemia such as by occlusion of the brachial artery by a blood pressure cuff) this will delay the reflectance (backward) wave which will then increase the overall width, W, of the pulse or decrease its height. FIG. 18 depicts with the indicated dotted line, the shift in the reflectance peak as a consequence of arterial diameter increases in a compliant artery. Both baseline and reactive PWF analysis are utilized herein to assess the functional status of the microvasculature.

In one embodiment of the invention, a smart Doppler sensor array module is provided that may be employed for PWF or PWF analysis. The smart Doppler sensor array module comprises an array of Doppler probes electrically coupled to a signal selection module that selects input from the probe delivering the strongest signal for recording. By the use of a smart Doppler sensor array, detection of the Doppler pulse is operator and individual anatomy independent. In one embodiment, such as that depicted in FIGS. 16A and B, the array 40 is disposed on the inside surface of blood pressure cuff 16 such that a plurality of detection sites over the brachial artery are provided. Leads 42 from the array 40 provide electrical communication with the controller 20. In one embodiment, the sensors are disposed in a local array as depicted in FIGS. 16A and B. In another embodiment the sensors are placed circumferentially around the cuff. In an alternative embodiment a separate sensor array such as that depicted in FIG. 16 C is utilized. The flow sensors are disposed in an array on patch, disk or pad 45. The patch can be self adhesive, manually head in place, or can further include a strap that goes circumferentially around the limb. In an alternative embodiment, the sensors are disposed in an essentially linear array that can be affixed around the arm or ankle like a strap. As depicted in FIG. 16A, a plurality of arrays may be employed. If any array is deployed over the radial artery and another over the brachial artery the arrays together can be used for PWV measurement.

The array may include sensors resonating at different frequencies providing information at different depths through a tissue. The array may further include sensors positioned at different angles for locating a maximum Doppler blood flow velocity. In one embodiment the target cardiovascular system is selected from the group consisting of: carotid, brachial, femoral, aortic and coronary.

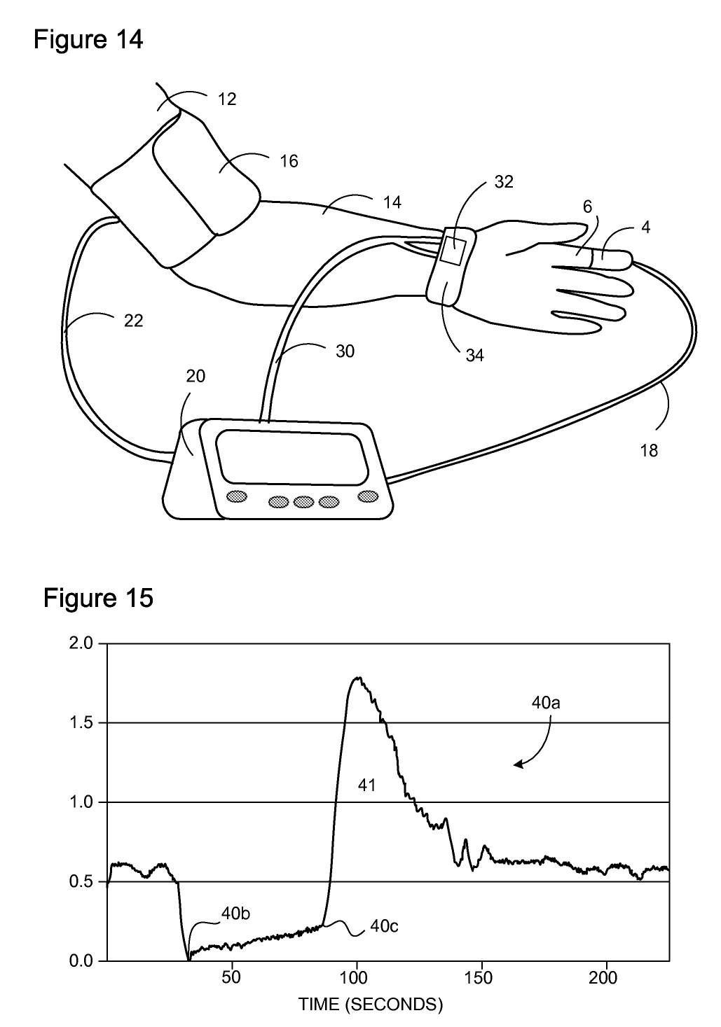

Doppler Flow Velocity Measurement (DFV): The present inventors have shown that continuous monitoring of Doppler Flow Velocity (DFV) before during and after inflation of a blood pressure cuff over the brachial artery provides measurement of vascular reactivity at either the radial or brachial levels. Methods and apparatus for comprehensive assessment of vascular function are provided by combining temperature changes with changes in peak systolic Doppler velocity measurement by Doppler ultrasonography. This combination of thermography and Doppler ultrasonography is herein termed “thermodoppler.” For example, and with an apparatus such as that as depicted in FIG. 14, the radial artery can be placed under continuous Doppler measurement together with fingertip or palm thermal monitoring before and after cuff occlusion test. In one embodiment, the probe is bidirectional Doppler probe 32 which is be placed over the radial artery and held in place by any number of attachments known in the art, including adhesives or, for example, a wrist band 34, and disposed to detect changes in flow velocity before during and after flow occlusion by use of a blood pressure cuff 16 disposed over the brachial artery on the upper arm 12. As depicted in FIG. 14, DFV readings are collected in processor 20. The relative position of a DFV sensor 32 over the radial artery in relation to a DTM sensor 4 on a finger tip is shown.

The results of a DFV response 40a is depicted in FIG. 15 is obtained by continuous monitoring of peak systolic Doppler velocity decreases after occlusion from its maximum immediately after release of the cuff (cuff deflation) and declining over time to base velocity before occlusion. This response inversely correlates with distal vascular resistance. The loss of flow with occlusion is depicted at 40b. When the cuff is released at 40c, resistance is minimum. Flow rapidly resumes and for a short period is greatly increased in a healthy individual as a consequence of dilation of the microvasculature. Upon reperfusion the resistance increases back to baseline resistance. The speed of return to baseline resistance, the area 41 under the produced curve as well as the slope, can be used to study the function of the resistant vasculature. Decreased vasodilative capacity (micovessels resume resistance quickly) after occlusion is indicative of inability of the vasculature to remain dilated and maintain high blood perfusion.

The results of this analysis (peak of the flow rebound, the slope of decline to baseline and the area under the curve) showed variability between individuals. DFW thus provides a measure of microvascular reactivity because it is the resistance vessels that establish whether flow can increase after release of the blood pressure cuff.

The Doppler flow velocity curve can be used as a non-invasive correlate of metabolic and biochemical factors affecting the distal microvascular resistance (e.g. lactate concentration, pH, calcium ion, etc. In summation, the curve can be calibrated to study, non-invasively, factors affecting vascular health.

Further Functional Testing Modalities: Specialized devices for performing one or more of the following techniques known to those of skill in the art may be added as diagnostic modules: skin color determination, nailbed capilloroscopy, ultrasound brachial artery imaging, forearm plethysmography, fingertip plethysmography, pulse oximetry, oxygen saturation change, pressure change, near-infrared spectroscopy measurements, peripheral artery tomometry, and combinations thereof. Optionally, an ankle-brachial blood pressure index can be determined for the subject. In one embodiment, various measurements of vascular reactivity are determined, weighted and a derivative composite index is determined.

In one embodiment, a combination of treadmill exercise test and one or more functional tests provided herein are designed to be superior to use of the exercise treadmill test alone in predicting the results of a nuclear test.

Serologic Testing Inputs: In one embodiment, the functional vascular status of the patient is considered together with additional diagnosis techniques in order to assess the subject's endothelial function. Additional diagnosis techniques may include one of more quantitative tests of the numbers and function of endothelial progenitor cells and related particles, such as endothelial derived microparticles in the peripheral blood. Determination of endothelial derived microparticles provides a measure of the degenerative status of the patient's endothelial system. Conversely, determination of numbers of Endothelial Precursor Stem Cells (EPC) in the peripheral blood provides a measure of the regenerative status of the patient's endothelial system. Assay of the status of circulatory progenitor cells and related elements are performed as baseline assessments and after stress provocation.

Other serologic tests include quantitative assays for one or more of the following factors: VEGF, VCAMI, ICAMI, Selectins such as soluble endothelium, leukocyte, and platelet selecting, VWF, CD54, c-reactive protein, homocysteine, Lp(a) and Lp-PLA₂. Further assays that may be employed include determination of: urinary albumin, serum fibrinogen, IL6, CD40/CD40L, serum amyloid A, PAI-1 test, t-PA test, homeostasis model assessment, white blood cell count, Neutrophil/lymphocyte ratio, platelet function tests, plasma and urinary level of asymmetrical (ADMA) and symmetrical (SDMA) dimethylarginine, exhaled nitric oxide, myelo-peroxidase (MPO), endothelin-1, thrombomodulin, tissue factor and tissue factor pathway inhibitor, markers of inflammation such as, for example, granulocyte-macrophage colony-stimulating factor (GM-CSF) and macrophage chemoattractant protein-1 (MCP-1), nitric oxide and its metabolites nitrates and nitrites, nitrosylated proteins, markers of oxidative stress including but not limited to free radical measurements of the blood or through the skin, TBAR, and/or extra cellular super oxide dismutase activity, and combinations thereof.

Risk Factor Analysis: In one embodiment, comprehensive vascular status of the patient is determined by considering the result of the functional macro, micro and neurovascular tests detailed herein together with risk factor determination including consideration of: BMI, body fat level, visceral fat, subcutaneous fat, glucose tolerance, fasting plasma glucose, blood insulin levels, HDL cholesterol, and fasting plasma insulin, as well as whether or not the patient is a smoker. The results of each assay are entered into a individual database for the patient and a combined relative risk factor calculated.

Structural Testing Inputs: In one embodiment, comprehensive vascular status of the patient is determined by considering the result of the functional macro, micro and neurovascular tests detailed herein together with additional structural diagnosis techniques as depicted in FIG. 4 in order to assess the subject's endothelial function. Additional diagnosis techniques may include one of more quantitative tests of the structural health of the vascular system including determining: coronary calcium score; carotid intima media thickness; MRI of the heart and brain, CT of the heart, intravascular optical coherent tomography; coronary fractional flow reserve; intravascular ultrasound radiofrequency backscatter analysis or Virtual Histology.

Further Vasostimulants: In alternative embodiments, in lieu of, or in addition to, using cuff occlusion for providing a vasostimulant, other vasostimulants may be employed while measuring both macro and micro vascular responses, and/or neurovascular responses: chemical vasostimulants such as nitroglycerin or transdermal substances, sympathetic mimetic agents, para-sympathetic mimetic agents, acetylcholine, vasodilating nitrates such as, for example, nitroprusside or glyceryl trinitrate, inhibitors of endothelium-derived contracting factors such as, for example, ACE inhibitors or angiotensin II receptor antagonists, cytoprotective agents such as, for example, free radical scavengers such as superoxide dismutase endothelium dependent agents such as, for example, acetylcholine, and/or endothelium independent agents such as, for example, nitroprusside or glycerin trinitrate, psychological vasostimulants such as aptitude tests, mental arithmetic, visual stimulation, physiological vasostimulants such as the Valsalva maneuver, a tilting test, physical exercise, whole body warming, whole body cooling, local warming, local cooling, contralateral handgrip, contralateral hand cooling, and painful stimuli such as, for example, nailbed compression, and a variety of others.

In an exemplary embodiment, the chemical vasostimulants may stimulate the vessel either through the endothelium or bypass the endothelium and directly affect the muscular part of the vessel wall, which is endothelium independent. In an exemplary embodiment, the vasostimulant may be, for example, a neuro-vasostimulant, a neurostimulant, a vasoconstrictor, a vasodialator, an endothermal layer stimulant, or a smooth muscle cell or medial layer stimulant. In an exemplary embodiment, a neuro-vasostimulant may include, for example, having the subject drink a glass of ice water.

Controlled Conditions: Skin microcirculation is divided into nutritional circulation and thermoregulatory circulation. It is well known that the thermoregulatory circulation that accounts for the majority of fingertip skin circulation is tightly controlled by autonomic nervous system. The thermoregulatory control mechanism is effected through arteriovenous shunts that bypass pre-capillary part of the side to the post-capillary of venous side. These networks of small arterioles are highly innervated and in cases of sympathetic stimuli such as mental stress and cold exposure, their contraction increase distal resistance and results in rerouting blood flow to AV shunts. This phenomenon explains cold fingers in fingertips during adrenergic stress. The side effect of this phenomenon on digital thermal monitoring of vascular reactivity (DTM) can be significant. However, such a “noise” effect is not limited to digital thermography. Indeed, studies have shown that BAUS is similarly affected by such sympathetic conditions. To minimize the effects of these conditions on endothelia function measurement, the International Task Force for Brachial Artery Reactivity has proposed certain guidelines for subject preparation and BAUS measurement to standardize the technique. Similar considerations can be exercised for DTM. However, the fact that this technique is much more simplified and can be repeated easily (potentially at the comfort home and ambulatory monitoring), makes it possible to have a more accurate assessment of endothelial function in those with hyperadrenergic conditions.

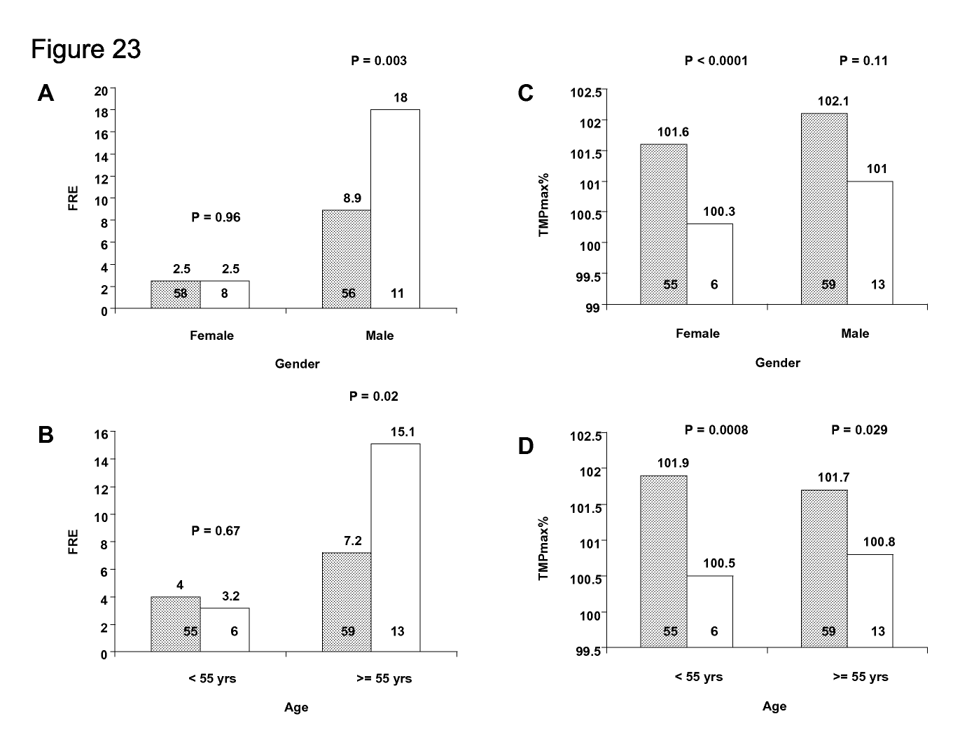

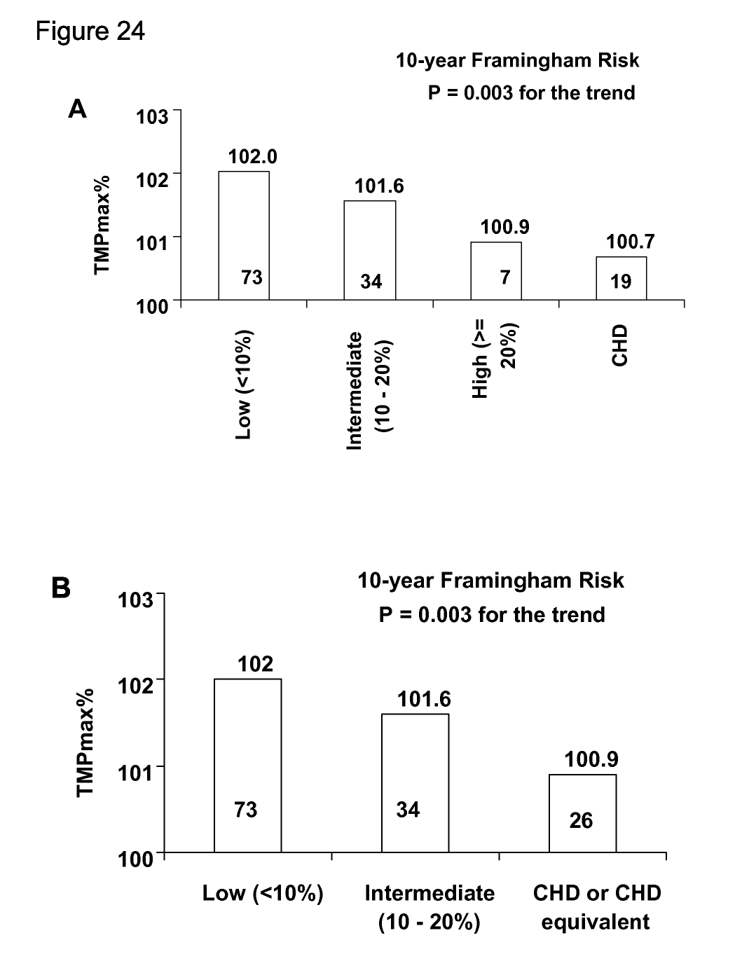

Relationship Between DTM and Cardiovascular Risk: Population-based cardiovascular risk calculators, e.g. Framingham Risk Estimation (FRE) are valuable in predicting long term future cardiovascular events in populations, but cannot accurately measure the status of vascular health in individuals. The present inventors developed DTM during reactive hyperemia as a complementary vascular function test to improve cardiovascular risk assessment. The ability of DTM ability to identify individuals with known coronary heart disease (CHD), and its correlation with FRE in a community setting was assessed. 133 individuals (51% male; 54±10 years; 19 with known CHD) underwent DTM measurements during 2 minutes of upper arm cuff occlusion. The results are depicted in FIG. 23A-D. Initial temperature and temperature fall were not significantly different in CHD vs. non-CHD, whereas DTM parameters of reactivity (temperature rebound and its slope) were consistently lower in subjects with CHD (p<0.006). As shown in FIG. 24, DTM discriminated between CHD and non-CHD more than FRE, particularly in women and in those ≦55 years. Significant inverse linear relationships were observed between DTM parameters and increasing CV risk, whether or not diabetes was considered a CHD equivalent, as illustrated in FIG. 24 for TMPₘₐₓ %. AUC in the ROC curve, with CHD as the response variable, were 0.6 for FRE (p<0.02), 0.71 for DTM (p<0.01), and 0.73 for DTM plus FRE (p<0.006). It was determined that DTM correlates with FRE and appears to better identify prevalent CHD, particularly in women and in younger individuals.

Relationship Between DTM and Metabolic Syndrome:

Endothelial dysfunction is the first stage of the atherosclerosis process and results in insulin resistance, metabolic syndrome (MS) and diabetes (DM). The ability of DTM, base on reactive hyperemia (RH), to identify metabolic status in asymptomatic at-risk adults was tested.

Study Population and Methods: 233 subjects (62% male, 58+11 yrs, 48% with family history of CHD, 46.1% hypertensive, 53% with hypercholesterolemia, 19% diabetic, and 38.6% smokers) were studied. Each underwent DTM during and after 5 min supra-systolic arm cuff inflation, CACS and FBS, Lipid profile, blood pressure, height, weight, waist and hip circumference measurements. Initial fingertip temperature at cuff inflation (TMPᵢ), lowest temperature (nadir) observed after cuff inflation (TMPₘᵢₙ), and indices of thermal recovery after cuff release (temperature rebound over baseline (TR) and slope of recovery) were measured.

Results: Room temperature was 74.6+2.7° F. TMPᵢ (90±4° F.) and TMPₘᵢₙ % (95.8±1.3° F.) were similar in three groups (p>0.7). TR % was (1.5±0.25° F.) in 94 with RRE<10% vs. (0.8±0.15° F.) in 75 with PRE>20% (p=0.01). 106 subjects with neither condition had higher TR % (2±0.23° F.) than 81 with MS (0.93±0.17° F.) and DM (0.91±0.2° F.) (p=0.001), suggesting reduced vascular reactivity in MS and DM and increasing PROCAM 10 year CHD risk (PRE %). After adjustment for age, gender and other CV risk factors by logistic regression, TR % remained significantly lower in the those with MS and DM than neither one (odds ratio=0.62 (95% CI 0.43-0.89, p=0.001)) and (odds ratio=0.68 (95% CI=0.52-0.88, p=0.003)) respectively also in PRE≧20% and CAC≧75% than PRE≦10% and CAC<10 (odds ratio=0.63 (95% CI=0.42-0.95, p=0.02)) and (odds ratio=0.57 (95% CI=0.35-0.92 p=0.01)) respectively. The data indicate that thermal/vascular function in the fingertip is associated inversely with presence of MS and DM also severity of CAC and PRE in an asymptomatic adults.

Relationship Between DTM and Coronary Calcium Score:

Comprehensive assessment of cardiovascular health must include measurement of risk factors as well as structural and functional evaluation of the vasculature. The ability of DTM to identify asymptomatic high risk individuals objectively defined by coronary artery calcium score (CACS)>75th percentile and 10y Framingham Risk Estimate (FRE)>15% was tested in the same population as the above mentioned Metabolic Syndrome study.

Results: TMPᵢ and TMPₘᵢₙ were not significantly different in high risk versus low risk groups (90.3±4.03 vs. 90.4±4.3° F., P>0.9) and (86.6±3.5 vs 86.4±3.8° F., P>0.6) respectively. In 105 subjects with FRE<5%, TR % was 1.57±0.23 vs. 0.84±0.14 in 52 with FRE>15% (p<0.01). TR % was also higher in 109 subjects with CACS<10 (1.82±0.19) vs. 62 with CACS≧75th percentile (1.09±0.22) (p<0.01), suggesting reduced vascular reactivity in both higher risk cohorts. After adjustment for age, gender and other traditional risk factors by logistic regression, TR % remained significantly lower in those with CACS≧75% than CACS<10 (odds ratio 0.57, 95% CI=0.35-0.92, p=0.02). Also TR % remained significantly lower in the those with FRE≧15% than FRE≦5% (odds ratio 0.57, 95%=CI 0.35-0.92, p<0.02) and those with metabolic syndrome than healthy population (odds ratio=0.62, 95% CI=0.43-0.89, P<0.001). The data indicate that vascular function measured by DTM during a 5-minute cuff occlusion reactive hyperemia test is inversely associated with the burden of atherosclerosis and risk factors of atherosclerosis as measured by CACS and FRE respectively.

It is understood that variations may be made in the foregoing without departing from the scope of the disclosed embodiments. Furthermore, the elements and teachings of the various illustrative embodiments may be combined in whole or in part some or all of the illustrated embodiments. Although illustrative embodiments have been shown and described, a wide range of modification, change and substitution is contemplated in the foregoing disclosure and in some instances, some features of the embodiments may be employed without a corresponding use of other features. Accordingly, it is appropriate that the appended claims be construed broadly and in a manner consistent with the scope of the embodiments disclosed herein.

We claim:

1. A method to improve risk assessment for an acute cardiovascular event in a patient presenting with chest pain comprising performing one or more vascular structural or functional tests on the patient.

2. The method of claim 1 wherein at least one vascular functional test is a non-invasive non-imaging vascular test that measures vascular response to reactive hyperemia induced by vascular occlusive challenge.

3. The method of claim 1 wherein at least one vascular functional test is selected from the group consisting of: DTM, BP, PWV, PWF, DFV, CLVR, ABI and combinations thereof.

4. The method of claim 1 wherein the vascular functional test is implemented using a modular functional vascular status assessment apparatus that comprises:
a CPU in electrical communication with and controlling a vascular function testing module including a digital thermal monitoring (DTM) module, a cuff management module, and a display or recorder.

5. The method of claim 4 wherein the modular functional vascular status assessment apparatus further comprises a Doppler module comprising at least one Doppler sensor.

6. The method of claim 4, wherein the DTM module comprises a plurality of temperature sensors.

7. The method of claim 4, wherein the cuff management module comprises a plurality of blood pressure cuffs and blood pressure detectors.

8. The method of claim 5, wherein the Doppler module controls a plurality of Doppler sensors.

9. The method of claim 5, wherein at least one Doppler sensor is adapted for measurement of Doppler flow velocity.

10. The method of claim 1, wherein the vascular functional test is a non-invasive coronary vasoreactivity imaging test that measures change in coronary flow and/or diameter with provocation.

11. The method of claim 10 wherein the provocation is a cold-pressor test.

12. The method of claim 1, further comprising determining a level of a marker of cardiovascular injury and/or a marker of atherosclerosis.

13. The method of claim 12, wherein the marker of cardiovascular injury comprises at least one marker selected from the group consisting of cardiac tropinins, CK-MB and CKMB isoforms, myoglobin, and the marker of cardiovascular risk comprises at least one marker selected from the group consisting of CRP (C reactive protein), I-CAM (intercellular adhesion molecule), SAP (serum amyloid P), MPO (myeloperoxidase), ADMA (asymmetric dimethylarginine), NO (nitric oxide), NO compounds/metabolites, and skin sterol.

14. The method of claim 1, wherein the vascular structural test provides a coronary calcium score.

15. The method of claim 11, wherein the vascular structural test measures a carotid artery intima-media thickness (IMT).

16. A method for identifying a high risk of an acute cardiovascular event in a patient presenting with chest pain comprising performing the following steps:
performing an EKG on the patient to determine an ST elevation;
if the ST is not elevated, performing a structural or functional atherosclerosis test on the patient to determine if further evaluation is required.

17. The method of claim 16, further comprising the step of determining a cardiovascular risk factor score for the patient.

18. A method for characterizing response to therapy in a clinical trial of a medication, device and/or drug comprising determining a micro or macro vascular function assessment for trial participants.

19. The method of claim 18, wherein the micro or macro vascular function assessment is a determined by one or more of the following tests: DTM, BP, PWV, PWF, DFV, CLVR, and ABI.

20. A method of risk assessment in a patient presenting with a possible acute cardiovascular symptom, comprising:
determining a cardiac specific injury marker level in the patient;
if the patient is negative or equivocal for cardiac specific injury markers, performing one or more structural or functional tests for atherosclerosis on the patient; and
triage the patients with negative cardiac injury markers based on the results of the one or more structural or functional tests for atherosclerosis.

21. A computer implemented method of risk assessment in a patient presenting with a possible acute cardiovascular symptom, comprising:
determining results from one or more vascular functional tests on the patient and placing the determined results of the one or more tests into a computational dataset corresponding to the patient;
receiving a status for each of a plurality of epidemiologic risk factors and inputting the received status of the epidemiologic risk factors into the computational dataset corresponding to the patient; and
computing a combined functional and epidemiologic relative risk for the individual from the dataset corresponding to the patient.

22. The computer implemented method of claim 21, wherein the one or more vascular function tests include one or more of: DTM, BP, PWV, PWF, DFV, CLVR, and ABI.

23. The computer implemented method of claim 21, further comprising:
receiving results from one or more structural assessments on the patient and placing the results of the one or more structural assessments into the computational dataset corresponding to the patient; and
computing a combined functional, epidemiologic, and structural relative risk for the individual from the dataset corresponding to the individual.

24. The computer implemented method of claim 23, wherein the structural assessments include determination of pathologic changes including one or more of: increased intima medial thickness, atherosclerotic plaque formation and calcium deposits in at least one vascular bed.
